(19) United States
(12) Patent Application Publication
(10) Pub. No.: US 2024/0342314 A1
(43) Pub. Date: Oct. 17, 2024

(54) SLC13A5 GENE THERAPY VECTORS AND USES THEREOF

(71) Applicant: THE BOARD OF REGENTS OF THE UNIVERSITY OF TEXAS SYSTEM, Austin, TX (US)

(72) Inventor: Rachel M BAILEY, Austin, TX (US)

(73) Assignee: THE BOARD OF REGENTS OF THE UNIVERSITY OF TEXAS SYSTEM, Austin, TX (US)

(21) Appl. No.: 18/683,101

(22) PCT Filed: Sep. 29, 2022
(86) PCT No.: PCT/US2022/077274

§ 371 Date: Feb. 12, 2024

Related U.S. Application Data
(60) Provisional application No. 63/250,761, filed on Sep. 30, 2021.
(60) Provisional application No. 63/364,655, filed on May 13, 2022.

(51) Int. Cl. A61K 48/00; A61P 25/28; C07K 14/705; C12N 15/86

(52) U.S. Cl. CPC A61K 48/0058; A61K 48/0075; A61P 25/28; C07K 14/705; C12N 15/86; C12N 2750/14143; C12N 2750/14145; C12N 2830/50

(57) ABSTRACT

The present disclosure provides methods and compositions for the treatment of diseases and genetic disorders linked to SLC13A5 loss, misfunction and/or deficiency, including neurological disorders, diseases, and conditions such as epileptic encephalopathy. The methods and compositions of the present disclosure comprise rAAV vectors and rAAV viral vectors comprising transgene nucleic acid molecules comprising nucleic acid sequences encoding for an SLC13A5 polypeptide.

### CROSS-REFERENCE TO RELATED APPLICATIONS

The present application claims the benefit of U.S. Provisional Patent Applications No. 63/250,761, filed Sep. 30, 2021 and No. 63/364,655, filed May 13, 2022, each of which is incorporated herein by reference in its entirety.

### INCORPORATION BY REFERENCE OF SEQUENCE LISTING

The Sequence Listing XML associated with this application is provided electronically in XML file format and is hereby incorporated by reference into the specification. The name of the XML file containing the Sequence Listing XML is “TAYS-015_SeqList_ST26.” The XML file is 61,095 bytes, created on Sep. 20, 2022, and is being submitted electronically via USPTO Patent Center.

### FIELD

The present disclosure relates generally to the field of gene therapy and in particular, to recombinant adeno-associated viral (AAV) vector particles (also known as rAAV viral vectors) comprising transgene sequences encoding SLC13A5 polypeptides, their manufacture, and their use to deliver transgenes to treat or prevent a disease or disorder, including diseases associated with loss, misfunction and/or deficiency of the SLC13A5 gene.

### BACKGROUND

Solute Carrier Family 13 member 5 (SLC13A5) is a high affinity sodium-dependent citrate cotransporter which mediates citrate entry into the cells. SLC13A5 is highly expressed in the in liver, teeth, testes, and brain, and mutations in the SLC13A5 gene are associated with neurological abnormalities (Yang et al., Child Neurology Open 2020,Vol. 7; 1-7). In particular, SLC13A5 deficiency causes autosomal-recessive epileptic encephalopathy in newborns and children, which manifests as early as in the first days of life and progresses into refractory epilepsy and development delay (Hardies et al, BRAIN 2015: 138; 3238-3250). To date, the treatment of SLC13A5 epilepsies is symptomatic, e.g., with anti-seizure medication. There is an unmet need for effective long-term treatments of SLC13A5 epilepsy.

### SUMMARY

The present disclosure provides recombinant adeno-associated virus (rAAV) vectors comprising in 5′ to 3′ direction: a) a first AAV ITR sequence; b) a promoter sequence; c) a transgene nucleic acid molecule, wherein the transgene nucleic acid molecule comprises a nucleic acid sequence encoding for an SLC13A5 polypeptide; d) a polyA sequence; and e) a second AAV ITR sequence.

In some aspects, a SLC13A5 polypeptide can comprise the amino acid sequence set forth in SEQ ID NO: 1.

In some aspects, a nucleic acid sequence encoding for a SLC13A5 polypeptide can be a codon optimized nucleic acid sequence encoding for a SLC13A5 polypeptide. In some aspects, an optimized nucleic acid sequence encoding for a SLC13A5 polypeptide can comprise the nucleic acid sequence set forth in SEQ ID NO: 3.

In some aspects, a codon optimized nucleic acid sequence encoding for a SLC13A5 polypeptide can exhibit at least 5%, at least 10%, at least 20%, at least 30%, at least 50%, at least 75%, at least 100%, at least 200%, at least 300%, at least 500%, or at least 1000% increased expression in a human subject relative to a wild-type or non-codon optimized nucleic acid sequence.

In some aspects, a first AAV ITR sequence can comprise the nucleic acid sequence set forth in SEQ ID NO: 7. In some aspects, a second AAV ITR sequence can comprise the nucleic acid sequence set forth in SEQ ID NO: 8.

In some aspects, a promoter sequence can comprise a Rous sarcoma virus (RSV) LTR promoter (optionally with an RSV enhancer), a cytomegalovirus (CMV) promoter, an SV40 promoter, a dihydrofolate reductase promoter, a beta-actin promoter, a phosphoglycerol kinase (PGK) promoter, a U6 promoter, a JetI promoter, an H1 promoter, a CAG promoter, a hybrid chicken beta-actin promoter, an MeCP2 promoter, an EF1 promoter, a ubiquitous chicken β-actin hybrid (CBh) promoter, a U1a promoter, a U1b promoter, an MeCP2 promoter, an MeP418 promoter, an MeP426 promoter, a minimal MeCP2 promoter, a VMD2 promoter, an mRho promoter, EFla promoter, Ubc promoter, human β-actin promoter, a synapsin (hSyn) promoter sequence, TRE promoter, Ac5 promoter, Polyhedrin promoter, CaMKIIa promoter, Gall promoter, TEF 1 promoter, GDS promoter, ADH1 promoter, Ubi promoter, or α-1-antitrypsin (hAAT) promoter. In some aspects, a promoter sequence can comprise the nucleic acid sequence set forth in SEQ ID NO: 21.

In some aspects, a polyA sequence comprises the nucleic acid sequence set forth in SEQ ID NO: 36.

The present disclosure provides rAAV vectors comprising, in the 5′ to 3′ direction: a) a first AAV ITR sequence comprising the nucleic acid sequence set forth in SEQ ID NO: 7; b) a promoter sequence comprising the nucleic acid sequence set forth in SEQ ID NO: 21; c) a transgene nucleic acid molecule, wherein the transgene nucleic acid molecule comprises a nucleic acid sequence encoding for an SLC13A5 polypeptide, wherein the nucleic acid sequence encoding for an SLC13A5 polypeptide comprises the nucleic acid sequence set forth in SEQ ID NO: 3; d) a polyA sequence comprising the nucleic acid sequence set forth in SEQ ID NO: 36; and e) a second AAV ITR sequence comprising the nucleic acid sequence set forth in SEQ ID NO: 8.

The present disclosure provides rAAV vectors comprising the nucleic acid sequence set forth in SEQ ID NO: 38.

The present disclosure provides rAAV viral vectors comprising: (i) an AAV capsid protein; and (ii) an rAAV vector of the present disclosure. In some aspects, an AAV capsid protein can be an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV 11 capsid protein, an AAV12 capsid protein, an AAV13 capsid protein, an AAVPHP.B capsid protein, an AAVrh74 capsid protein or an AAVrh.10 capsid protein. In some aspects, an AAV capsid protein can be an AAV9 capsid protein.

The present disclosure provides a pharmaceutical composition comprising: a) the rAAV viral vector of the present disclosure; and at least one pharmaceutically acceptable excipient and/or additive.

The present disclosure provides methods for treating a subject having a disease and/or disorder involving an SLC13A5 gene, the method comprising administering to the subject at least one therapeutically effective amount of a rAAV viral vector or pharmaceutical composition of the present disclosure.

The present disclosure provides the rAAV viral vectors or the pharmaceutical composition of the present disclosure for use in treating a disease and/or disorder involving an SLC13A5 gene in a subject in need thereof.

In some aspects, a disease and/or disorder involving an SLC13A5 gene can be neonatal epileptic encephalopathy.

In some aspects, an rAAV viral vector or pharmaceutical composition of the present disclosure can be administered to a subject at a dose ranging from about 10¹¹ to about 10¹⁸ vector genomes.

In some aspects, an rAAV viral vector or pharmaceutical composition of the present disclosure can be administered to a subject at a dose ranging from about 10¹³ to about 10¹⁶ vector genomes.

In some aspects, the rAAV viral vector or the pharmaceutical composition is administered to the subject at a dose of about 2×10¹¹ or about 8×10¹¹ vector genomes.

In some aspects, an rAAV viral vector or pharmaceutical composition of the present disclosure can be administered to a intravenously, intrathecally, intracisterna-magna, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally or intranerve.

In some aspects, an rAAV viral vector or pharmaceutical composition of the present disclosure can be administered intrathecally.

In some aspects, an rAAV viral vector or pharmaceutical composition of the present disclosure can be administered intracisterna-magna.

In another aspect, provided herein is a recombinant adeno-associated virus (rAAV) vector comprising in 5′ to 3′ direction: (a) a first AAV ITR sequence comprising the sequence of SEQ ID NO: 7; (b) a promoter sequence; (c) a nucleic acid sequence encoding an SLC13A5 polypeptide, wherein the SLC13A5 polypeptide comprises the amino acid sequence set forth in SEQ ID NO: 1; (d) a polyA sequence; and (e) a second AAV ITR sequence comprising the sequence of SEQ ID NO: 8.

In some embodiments, the nucleic acid sequence encoding the SLC13A5 polypeptide is a codon optimized nucleic acid sequence. In some embodiments, the codon optimized nucleic acid sequence encoding a SLC13A5 polypeptide comprises the nucleic acid sequence set forth in SEQ ID NO: 3.

In some embodiments, the promoter sequence comprises the nucleic acid sequence set forth in SEQ ID NO: 21.

In some embodiments, the polyA sequence comprises the nucleic acid sequence set forth in SEQ ID NO: 36.

In some embodiments, the rAAV vector comprises the nucleic acid sequence set forth in SEQ ID NO: 38.

In another aspect, provided herein is an rAAV viral vector comprising: (i) an AAV capsid protein; and (ii) an rAAV vector provided herein. In some embodiments, the AAV capsid protein is an AAV9 capsid protein.

In another aspect, provided herein is a pharmaceutical composition comprising an rAAV viral vector provided herein and at least one pharmaceutically acceptable excipient and/or additive.

In another aspect, provided herein is a method for treating a subject having a disease and/or disorder involving an SLC13A5 gene, the method comprising administering to the subject at least one therapeutically effective amount of an rAAV viral vector or a pharmaceutical composition provided herein. In some embodiments, the disease and/or disorder involving an SLC13A5 gene is neonatal epileptic encephalopathy. In some embodiments, the rAAV viral vector or pharmaceutical composition is administered intrathecally. In some embodiments, the rAAV viral vector or pharmaceutical composition is administered intracisterna-magna.

### BRIEF DESCRIPTION OF THE DRAWINGS

FIG. 1 shows the survival of WT C57BL/6J mice intravenously treated with 1×10¹⁴ vg/kg AAV9/hSLC13A5 at 8 weeks of age.

FIGS. 2A and 2B show body weight of mice treated with AAV9/hSLC13A5. C57BL/6J mice were untreated or dosed with 1×10⁴ vg/kg AAV9/hSLC13A5 via a tail vein injection at 8 weeks of age. Mice were weighed 3 times per week for the first 8 weeks, then one time per week until 9 months post-injection and then monthly thereafter. FIG. 2A shows data for female mice, FIG. 2B shows data for male mice. Data shown as Mean±SEM.

FIGS. 3A-3E show blood chemistry results 8 weeks post-dosing. Shown are levels of total bilirubin (FIG. 3A), aspartate aminotransferase (FIG. 3B), albumin (FIG. 3C), blood urea nitrogen (FIG. 3D) and creatine kinase (FIG. 3E). *p<0.05, student's unpaired t-test. Data shown as Mean SEM.

FIG. 4 shows survival of WT C57BL/6J mice treated with intrathecally a dose of 8×10¹¹ vg of AAV9/hSLC13A5 at 8 weeks of age.

FIGS. 5A and 5B show body weight of mice treated with AAV9/hSLC13A5. C57BL/6J mice were intrathecally injected with either vehicle or 8×10¹¹ vg AAV9/hSLC13A5 at 8 weeks of age. Mice were weighed 3 times per week for the first 8 weeks, then one time per week until 6 months post-injection and then monthly thereafter. FIG. 5A shows data from female mice, FIG. 5B shows data from male mice. Data shown as Mean±SEM.

FIGS. 6A-6E show blood chemistry results at study endpoint. Shown are levels of total bilirubin (FIG. 6A), albumin (FIG. 6B), aspartate aminotransferase (FIG. 6C), blood urea nitrogen (FIG. 6D) and creatine kinase (FIG. 6E). Data shown as Mean±SEM.

FIG. 7 shows survival of mice treated intrathecally with a low (2×10¹¹ vg) or a high (8×10¹¹ vg) dose of AAV9/hSLC13A5.

FIGS. 8A and 8B show body weight of mice treated with AAV9/hSLC13A5. WT C57BL/6J mice were intrathecally injected with either vehicle or 2×10¹¹ vg (low dose) or 8×10¹¹ vg (high dose) AAV9/hSLC13A5. Mice were weighed 3 times per week for the first 8 weeks, then one time per week thereafter. FIG. 8A shows data from female mice, FIG. 8B shows data from male mice. Data shown as Mean±SEM.

FIGS. 9A-9J show blood chemistry results at 8 weeks post-dosing (9A-9E) and at 12 months post-injection (9F-9J) in WT C57BL/6J mice. Shown are total bilirubin (FIGS. 9A and 9F), albumin (FIGS. 9B and 9G), aspartate aminotransferase (FIGS. 9C and 9H), blood urea nitrogen (FIGS. 9D and 9I) and creatine kinase (FIGS. 9E and 9J). **p<0.01, One-way ANOVA, Tukey's post-hoc analysis.

FIG. 10 is a graph depicting plasma citrate levels of KO mice relative to WT control littermates 4 weeks post-injection with either vehicle or 2×1011 vg (low dose (LD)) or 8×1011 vg (high dose (HD)) AAV9/hSLC13A5 via intrathecal (IT) administration. Vehicle treated KO mice have elevated citrate levels relative to WT mice, which is significantly decreased in a dose-dependent manner in treated mice. *p<0.05, ****p<0.0001, One-way ANOVA, Tukey's post-hoc analysis.

FIG. 11A and FIG. 11B are graphs depicting electroencephalogram (EEG) recorded brain activity in wild-type and KO mice. At post-natal day 10 (P10), mice were treated with vehicle, 2×10¹¹ vg (low dose (LD)) or 8×10¹¹ vg (high dose (HD)) AAV9/hSLC13A5 via intrathecal (IT) administration. At 3 months of age (3 mo), mice were treated with vehicle or HD via IT or intra-cisterna magna (ICM) administration. FIG. 11A depicts number of spike trains in P10 mice assessed at 3 months of age. FIG. 11B depicts number of spike trains in 3 month treated group assessed at 8 months of ages.

FIGS. 12A-12D are graphs depicting average activity in wild-type and KO mice. Mice were treated at P10 with vehicle, 2×10¹¹ vg (low dose (LD)) or 8×10¹¹ vg (high dose (HD)) AAV9/hSLC13A5 via intrathecal (IT) administration. FIG. 12A depicts average activity during light cycle/sleep periods for WT and KO mice treated with vehicle. FIG. 12B depicts average activity during light cycle/sleep periods for KO mice treated with vehicle, LD, or HD. FIG. 12C depicts average activity during dark cycle/awake periods for WT and KO mice treated with vehicle. FIG. 12D depicts average activity during dark cycle/awake periods for KO mice treated with vehicle, LD, or HD. P10: n=17-22/group. *p<0.05, **p<0.01.

FIG. 13A and FIG. 13B are graphs depicting percent survival in KO and WT mice following repeated injections of pentylenetetrazol to test seizure susceptibility. Mice were treated with vehicle, 2×1011 vg (low dose (LD)) or 8×1011 vg (high dose (HD)) AAV9/hSLC13A5 via intrathecal (IT) or intra-cisterna magna (ICM) administration. FIG. 13A depicts mice treated at P10 tested at 4 months of age. FIG. 13B depicts mice treated at 3 month old tested at 9 months of ages.

FIGS. 14A-14D are graphs depicting seizure severity and susceptibility to pentylenetetrazol. Mice were treated with vehicle, 2×10¹¹ vg (low dose (LD)) or 8×10¹¹ vg (high dose (HD)) AAV9/hSLC13A5 via intrathecal (IT) or intra-cisterna magna (ICM) administration at P10 or 3 months of age. Seizure severity was measured via the Modified Racine scoring scale in P10 treated mice (FIG. 14A) or 3 month-old treated mice (FIG. 14B). Latency to seizure was evaluated in P10 treated mice (FIG. 14C) and 3 month-old treated mice (FIG. 14D). *p<0.05, **p<0.01, Two-way ANOVA, Sidak's post-hoc analysis.

FIG. 15A is a graph depicting vector biodistribution in P10 treated knockout mice. Mice were treated with vehicle, 2×10¹¹ vg (low dose (LD)) or 8×10¹¹ vg (high dose (HD)) AAV9/hSLC13A5 via intrathecal (IT) administration.

FIG. 15B is a graph depicting vector biodistribution in 3 month old treated knockout mice. Mice were treated with vehicle or 8×10¹¹ vg (high dose (HD)) AAV9/hSLC13A5 via intrathecal (IT) or intra-cisterna magna (ICM) administration.

FIG. 16 is a series of images depicting vector-delivered SLC13A5 expression in the brain of P10 or 3 month-old treated mice. Mice were treated with vehicle, 2×10¹¹ vg (low dose (LD)) or 8×10¹¹ vg (high dose (HD)) AAV9/hSLC13A5 via intrathecal (IT) or intra-cisterna magna (ICM) administration.

### DETAILED DESCRIPTION

The present disclosure provides, inter alia, isolated polynucleotides, recombinant adeno-associated virus (rAAV) vectors, and rAAV viral vectors comprising transgene nucleic acid molecules comprising nucleic acid sequences encoding for Solute Carrier Family 13 member 5 (SLC13A5) polypeptides. The present disclosure also provides methods of manufacturing these isolated polynucleotides, rAAV vectors, and rAAV viral vectors, as well as their use to deliver transgenes to treat or prevent a disease or disorder, including diseases associated with loss, misfunction and/or deficiency of an SLC13A5 gene.

The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3: 1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types. At least 11 sequentially numbered AAV serotypes are known in the art. Non-limiting exemplary serotypes useful in the methods disclosed herein include any of the 11 serotypes, e.g., AAV2, AAV8, AAV9, or variant serotypes, e.g., AAV-DJ and AAV PHP.B. The AAV particle comprises, consists essentially of, or consists of three major viral proteins: VP1, VP2 and VP3. In some aspects, the AAV refers to the serotype AAV1, AAV2, AAV4, AAV5, AAV6, AAV7, AAV8, AAV9, AAV10, AAV11, AAV12, AAV13, AAVPHP.B, AAVrh74 or AAVrh.10.

Exemplary adeno-associated viruses and recombinant adeno-associated viruses include, but are not limited to all serotypes (e.g., AAV1, AAV2, AAV3, AAV4, AAV5, AAV6, AAV7, AAV8, AAV9, AAV10, AAV11, AAV12, AAV13, AAVPHP.B, AAVrh74 and AAVrh.10). Exemplary adeno-associated viruses and recombinant adeno-associated viruses include, but are not limited to, self-complementary AAV (scAAV) and AAV hybrids containing the genome of one serotype and the capsid of another serotype (e.g., AAV2/5, AAV-DJ and AAV-DJ8). Exemplary adeno-associated viruses and recombinant adeno-associated viruses include, but are not limited to, rAAV-LK03, AAV-KP-1 (described in detail in Kerun et al. JCI Insight, 2019; 4(22):e131610) and AAV-NP59 (described in detail in Paulk et al. Molecular Therapy, 2018; 26(1): 289-303).

### AAV Structure and Function

AAV is a replication-deficient parvovirus, the single-stranded DNA genome of which is about 4.7 kb in length, including two 145-nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_001862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in U.S. Pat. No. 9,434,928. U.S. Pat. No. 9,434,928 also provides the sequences of the capsid proteins and a self-complementary genome. In one aspect, an AAV genome is a self-complementary genome. Cis-acting sequences directing viral DNA replication (rep), encapsidation/packaging, and host cell chromosome integration are contained within AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome.

The cap gene is expressed from the p40 promoter and encodes the three capsid proteins, VP1, VP2, and VP3. Alternative splicing and non-consensus translational start sites are responsible for the production of the three related capsid proteins. More specifically, after the single mRNA from which each of the VP1, VP2 and VP3 proteins are translated is transcribed, it can be spliced in two different manners: either a longer or shorter intron can be excised, resulting in the formation of two pools of mRNAs: a 2.3 kb- and a 2.6 kb-long mRNA pool. The longer intron is often preferred and thus the 2.3-kb-long mRNA can be called the major splice variant. This form lacks the first AUG codon, from which the synthesis of VP1 protein starts, resulting in a reduced overall level of VP1 protein synthesis. The first AUG codon that remains in the major splice variant is the initiation codon for the VP3 protein. However, upstream of that codon in the same open reading frame lies an ACG sequence (encoding threonine) which is surrounded by an optimal Kozak (translation initiation) context. This contributes to a low level of synthesis of the VP2 protein, which is actually the VP3 protein with additional N terminal residues, as is VP1, as described in Becerra S P et al., (December 1985). “Direct mapping of adeno-associated virus capsid proteins B and C: a possible ACG initiation codon”.

Proceedings of the National Academy of Sciences of the United States of America. 82 (23): 7919-23, Cassinotti P et al., (November 1988). “Organization of the adeno-associated virus (AAV) capsid gene: mapping of a minor spliced mRNA coding for virus capsid protein 1”. Virology. 167 (1): 176-84, Muralidhar S et al., (January 1994). “Site-directed mutagenesis of adeno-associated virus type 2 structural protein initiation codons: effects on regulation of synthesis and biological activity”. Journal of Virology. 68 (1): 170-6, and Trempe J P, Carter B J (September 1988). “Alternate mRNA splicing is required for synthesis of adeno-associated virus VP1 capsid protein”. Journal of Virology. 62 (9): 3356-63, each of which is herein incorporated by reference. A single consensus polyA site is located at map position 95 of the AAV genome. The life cycle and genetics of AAV are reviewed in Muzyczka, Current Topics in Microbiology and Immunology, 158: 97-129 (1992).

Each VP1 protein contains a VP1 portion, a VP2 portion and a VP3 portion. The VP1 portion is the N-terminal portion of the VP1 protein that is unique to the VP1 protein. The VP2 portion is the amino acid sequence present within the VP1 protein that is also found in the N-terminal portion of the VP2 protein. The VP3 portion and the VP3 protein have the same sequence. The VP3 portion is the C-terminal portion of the VP1 protein that is shared with the VP1 and VP2 proteins.

The VP3 protein can be further divided into discrete variable surface regions I-IX (VR-I-IX). Each of the variable surface regions (VRs) can comprise or contain specific amino acid sequences that either alone or in combination with the specific amino acid sequences of each of the other VRs can confer unique infection phenotypes (e.g., decreased antigenicity, improved transduction and/or tissue-specific tropism relative to other AAV serotypes) to a particular serotype as described in DiMatta et al., “Structural Insight into the Unique Properties of Adeno-Associated Virus Serotype 9” J. Virol., Vol. 86 (12): 6947-6958, June 2012, the contents of which are incorporated herein by reference.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate AAV vectors. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics. Recombinant AAV (rAAV) genomes of the invention comprise, consist essentially of, or consist of a nucleic acid molecule encoding a therapeutic protein (e.g., SLC13A5) and one or more AAV ITRs flanking the nucleic acid molecule. Production of pseudotyped rAAV is disclosed in, for example, WO2001083692. Other types of rAAV variants, for example rAAV with capsid mutations, are also contemplated. See, e.g., Marsic et al., Molecular Therapy, 22(11): 1900-1909 (2014). The nucleotide sequences of the genomes of various AAV serotypes are known in the art.

### Isolated Polynucleotides Comprising Transgene Sequences

The present disclosure provides isolated polynucleotides comprising at least one transgene nucleic acid molecule.

In some aspects, a transgene nucleic acid molecule can comprise a nucleic acid sequence encoding an SLC13A5 polypeptide, or at least one fragment thereof. In some aspects, a transgene nucleic acid molecule can comprise a nucleic acid sequence encoding a biological equivalent of an SLC13A5 polypeptide.

In some aspects, an SLC13A5 polypeptide comprises, consists essentially of, or consists of an amino acid sequence at least 65%, 70%, 75%, 80%, 85%, 90%, 95%, 96%, 97%, 98%, 99% or 100% (or any percentage in between) identical to the amino acid sequence set forth in SEQ ID NO: 1, or a fragment thereof. In some aspects, an SLC13A5 polypeptide comprises, consists essentially of, or consists of an amino acid sequence at least 65%, 70%, 75%, 80%, 85%, 90%, 95%, 96%, 97%, 98%, 99% or 100% (or any percentage in between) identical to at least one portion of the amino acid sequence set forth in SEQ ID NO: 2, or a fragment thereof. In some embodiments, the fragment is a functional fragment, e.g., a fragment that retains at least one function of wildtype SLC13A5.

In some aspects, a nucleic acid sequence encoding an SLC13A5 polypeptide comprises, consists essentially of, or consists of a nucleic acid sequence at least 65%, 70%, 75%, 80%, 85%, 90%, 95%, 96%, 97%, 98%, 99% or 100% (or any percentage in between) identical to the nucleic acid sequence set forth in SEQ ID NO: 2.

In some aspects, the nucleic acid sequence encoding an SLC13A5 polypeptide can be a codon optimized nucleic acid sequence that encodes an SLC13A5 polypeptide. A codon optimized nucleic acid sequence encoding an SLC13A5 polypeptide can comprise, consist essentially of, or consist of a nucleic acid sequence that is no more than 65%, 70%, 75%, 80%, 85%, 90%, 95%, 96%, 97%, 98%, 99% (or any percentage in between) identical to the wildtype human nucleic acid sequence encoding the SLC13A5 polypeptide. As used herein, the “wildtype human nucleic acid sequence encoding the SLC13A5 polypeptide” refers to the nucleic acid sequence that encodes the SLC13A5 polypeptide in a human genome. Exemplary wildtype human nucleic acid sequences encoding the SLC13A5 peptide is set forth in SEQ ID NOs: 4-6. An exemplary wildtype SLC13A5 polypeptide is set forth in SEQ ID NO: 2. An exemplary codon optimized sequence encoding SLC13A5 is set forth in SEQ ID NO: 3.

In some aspects, a codon optimized nucleic acid sequence encoding an SLC13A5 polypeptide, such as those put forth in SEQ ID NOs: 1 or 2, can comprise no donor splice sites. In some aspects, a codon optimized nucleic acid sequence encoding an SLC13A5 polypeptide can comprise no more than about one, or about two, or about three, or about four, or about five, or about six, or about seven, or about eight, or about nine, or about ten donor splice sites. In some aspects, a codon optimized nucleic acid sequence encoding an SLC13A5 polypeptide comprises at least one, or at least two, or at least three, or at least four, or at least five, or at least six, or at least seven, or at least eight, or at least nine, or at least ten fewer donor splice sites as compared to the wildtype human nucleic acid sequence encoding the SLC13A5 polypeptide. Without wishing to be bound by theory, the removal of donor splice sites in the codon optimized nucleic acid sequence can unexpectedly and unpredictably increase expression of the SLC13A5 polypeptide in vivo, as cryptic splicing is prevented. Moreover, cryptic splicing may vary between different subjects, meaning that the expression level of the SLC13A5 polypeptide comprising donor splice sites may unpredictably vary between different subjects.

In some aspects, a codon optimized nucleic acid sequence encoding an SLC13A5 polypeptide, such as those put forth in SEQ ID NOs: 1 or 2, can have a GC content that differs from the GC content of the wildtype human nucleic acid sequence encoding the SLC13A5 polypeptide. In some aspects, the GC content of a codon optimized nucleic acid sequence encoding an SLC13A5 polypeptide is more evenly distributed across the entire nucleic acid sequence, as compared to the wildtype human nucleic acid sequence encoding the SLC13A5 polypeptide. Without wishing to be bound by theory, by more evenly distributing the GC content across the entire nucleic acid sequence, the codon optimized nucleic acid sequence exhibits a more uniform melting temperature (“Tm”) across the length of the transcript. The uniformity of melting temperature results unexpectedly in increased expression of the codon optimized nucleic acid in a human subject, as transcription and/or translation of the nucleic acid sequence occurs with less stalling of the polymerase and/or ribosome.

In some aspects, the codon optimized nucleic acid sequence encoding an SLC13A5 polypeptide, such as those put forth in SEQ ID NOs: 1 or 2, exhibits at least 5%, at least 10%, at least 20%, at least 30%, at least 50%, at least 75%, at least 100%, at least 200%, at least 300%, at least 500%, or at least 1000% increased expression in a human subject relative to a wild-type or non-codon optimized nucleic acid sequence encoding an SLC13A5 polypeptide.

In some aspects, an SLC13A5 polypeptide can further comprise a protein tag. Without wishing to be bound by theory, the inclusion of a protein tag can allow for the detection and/or visualization of the exogenous SLC13A5 polypeptide. As would be appreciated by the skilled artisan, non-limiting examples of protein tags include Myc tags, poly-histidine tags, FLAG-tags, HA-tags, SBP-tags or any other protein tag known in the art.

### AAV Vectors

In some aspects, the isolated polynucleotides comprising at least one transgene nucleic acid molecule described herein can be a recombinant AAV (rAAV) vector.

As used herein, the term “vector” refers to a nucleic acid comprising, consisting essentially of, or consisting of an intact replicon such that the vector may be replicated when placed within a cell, for example by a process of transfection, infection, or transformation. It is understood in the art that once inside a cell, a vector may replicate as an extrachromosomal (episomal) element or may be integrated into a host cell chromosome. Vectors may include nucleic acids derived from retroviruses, adenoviruses, herpesvirus, baculoviruses, modified baculoviruses, papovaviruses, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising, consisting essentially of, or consisting of DNA condensed with cationic polymers such as heterogeneous polylysine, defined-length oligopeptides, and polyethyleneimine, in some cases contained in liposomes; and the use of ternary complexes comprising, consisting essentially of, or consisting of a virus and polylysine-DNA.

With respect to general recombinant techniques, vectors that contain both a promoter and a cloning site into which a polynucleotide can be operatively linked are well known in the art. Such vectors are capable of transcribing RNA in vitro or in vivo, and are commercially available from sources such as Agilent Technologies (Santa Clara, Calif) and Promega Biotech (Madison, Wis.). In order to optimize expression and/or in vitro transcription, it may be necessary to remove, add or alter 5′ and/or 3′ untranslated portions of cloned transgenes to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

An “rAAV vector” as used herein refers to a vector comprising, consisting essentially of, or consisting of one or more transgene nucleic acid molecules and one or more AAV inverted terminal repeat sequences (ITRs). Such AAV vectors can be replicated and packaged into infectious viral particles when present in a host cell that provides the functionality of rep and cap gene products; for example, by transfection of the host cell. In some aspects, AAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the AAV vector genome. Plasmids containing rAAV vectors may also contain elements for manufacturing purposes, e.g., antibiotic resistance genes, origin of replication sequences etc., but these are not encapsidated and thus do not form part of the AAV particle.

In some aspects, an rAAV vector can comprise at least one transgene nucleic acid molecule. In some aspects, an rAAV vector can comprise at least one AAV inverted terminal (ITR) sequence. In some aspects, an rAAV vector can comprise at least one promoter sequence. In some aspects, an rAAV vector can comprise at least one enhancer sequence. In some aspects, an rAAV vector can comprise at least one polyA sequence. In some aspects, an rAAV vector can comprise a RepCap sequence.

In some aspects, an rAAV vector can comprise a first AAV ITR sequence, a promoter sequence, a transgene nucleic acid molecule and a second AAV ITR sequence. In some aspects, an rAAV vector can comprise, in the 5′ to 3′ direction, a first AAV ITR sequence, a promoter sequence, a transgene nucleic acid molecule and a second AAV ITR sequence.

In some aspects, an rAAV vector can comprise a first AAV ITR sequence, a promoter sequence, a transgene nucleic acid molecule, a polyA sequence and a second AAV ITR sequence. In some aspects, an rAAV vector can comprise, in the 5′ to 3′ direction, a first AAV ITR sequence, a promoter sequence, a transgene nucleic acid molecule, a polyA sequence and a second AAV ITR sequence.

In some aspects, an rAAV vector can comprise more than one transgene nucleic acid molecule.

In some aspects, an rAAV vector can comprise at least two transgene nucleic acid molecules, such that the rAAV vector comprises a first transgene nucleic acid molecule and an at least second transgene nucleic acid molecule. In some aspects, the first and the at least second transgene nucleic acid molecule can comprise the same nucleic acid sequence. In some aspects, the first and the at least second transgene nucleic acid molecules can comprise different nucleic acid sequences. In some aspects, the first and the at least second transgene nucleic acid sequences can be adjacent to each other.

In some aspects, an rAAV vector can comprise more than one promoter sequence. In some aspects, an rAAV vector can comprise at least two promoter sequences, such that the rAAV vector comprises a first promoter sequence and an at least second promoter sequence. In some aspects, the first and the at least second promoter sequences can comprise the same sequence. In some aspects, the first and the at least second promoter sequences can comprise different sequences. In some aspects, the first and the at least second promoter sequences can be adjacent to each other. In some aspects wherein an rAAV vector also comprises a first transgene nucleic acid molecule and an at least second transgene nucleic acid molecule, the first promoter can be located upstream (5′) of the first transgene nucleic acid molecule and the at least second promoter can be located between the first transgene nucleic acid molecule and the at least second transgene nucleic acid molecule, such that the at least second promoter is downstream (3′) of the first transgene nucleic acid molecule and upstream (5′) of the at least second transgene nucleic acid molecule.

Any of the preceding rAAV vectors can further comprise at least one enhancer. The at least one enhancer can be located anywhere in the rAAV vector. In some aspects, the at least one enhancer can be located immediately upstream (5′) of a promoter. Thus, an rAAV vector can comprise, in the 5′ to 3′ direction, a first AAV ITR sequence, an enhancer, a promoter sequence, a transgene nucleic acid molecule, a polyA sequence, and a second AAV ITR sequence. In some aspects, the at least one enhancer can be located immediately downstream (3′) of a promoter. Thus, an rAAV vector can comprise, in the 5′ to 3′ direction, a first AAV ITR sequence, a promoter sequence, an enhancer, a transgene nucleic acid molecule, a polyA sequence, and a second AAV ITR sequence. In some aspects, the at least one enhancer can be located immediately downstream of a transgene nucleic acid molecule. Thus, an rAAV vector can comprise, in the 5′ to 3′ direction, a first AAV ITR sequence, a promoter sequence, a transgene nucleic acid molecule, an enhancer, a polyA sequence, and a second AAV ITR sequence.

### AAV ITR Sequences

In some aspects, an AAV ITR sequence can comprise any AAV ITR sequence known in the art. In some aspects, an AAV ITR sequence can be an AAV1 ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAV 11 ITR sequence, an AAV12 ITR sequence, an AAV13 ITR sequence, an AAVrh74 ITR sequence or an AAVrh.10 ITR sequence.

Thus, in some aspects, an AAV ITR sequence can comprise, consist essentially of, or consist of an AAV1 ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAV11 ITR sequence, an AAV12 ITR sequence, an AAV13 ITR sequence, an AAVrh74 ITR sequence, or an AAVrh.10 ITR sequence. In some embodiments, an AAV ITR sequence is a wildtype AAV ITR sequence. In some embodiments, an AAV ITR sequence is modified (e.g., mutated) AAV ITR sequence. In some embodiments, an rAAV vector described herein comprises one mutated AAV ITR and one wildtype AAV ITR.

In some aspects, an AAV ITR can comprise consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in any one of SEQ ID NOs: 7-18.

In some aspects, an AAV ITR can comprise consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO: 7.

In some aspects, an AAV ITR can comprise consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO: 8.

In some aspects, an rAAV provided herein comprises a first and a second AAV ITR sequence, wherein the first AAV ITR sequence comprises, consists essentially of, or consists of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO: 7 and the second AAV ITR sequence comprises, consists essentially of, or consists of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO: 8.

### Promoter Sequence and Enhancers

The term “promoter” and “promoter sequence” as used herein means a control sequence that is a region of a polynucleotide sequence at which the initiation and rate of transcription of a coding sequence, such as a gene or a transgene, are controlled. Promoters may be constitutive, inducible, repressible, or tissue-specific, for example. Promoters may contain genetic elements at which regulatory proteins and molecules such as RNA polymerase and transcription factors may bind. Non-limiting exemplary promoters include Rous sarcoma virus (RSV) LTR promoter (optionally with the RSV enhancer), a cytomegalovirus (CMV) promoter, an SV40 promoter, a dihydrofolate reductase promoter, a β-actin promoter, a phosphoglycerol kinase (PGK) promoter, a U6 promoter, a synapsin promoter, an H1 promoter, a ubiquitous chicken β-actin hybrid (CBh) promoter, a small nuclear RNA (U1a or U1b) promoter, an MECP2 promoter, an MeP418 promoter, an MeP426 promoter, a human variant of the MeP426 promoter, a minimal MECP2 promoter, a VMD2 promoter, an mRho promoter, or an EF1 promoter.

Additional non-limiting exemplary promoters provided herein include, but are not limited to EFla, Ubc, human β-actin, CAG, TRE, Ac5, Polyhedrin, CaMKIIa, Gall, TEF1, GDS, ADH1, Ubi, and α-1-antitrypsin (hAAT). It is known in the art that the nucleotide sequences of such promoters may be modified in order to increase or decrease the efficiency of mRNA transcription. See, e.g., Gao et al. (2018) Mol. Ther.: Nucleic Acids 12:135-145 (modifying TATA box of 7SK, U6 and H1 promoters to abolish RNA polymerase III transcription and stimulate RNA polymerase II-dependent mRNA transcription). Synthetically-derived promoters may be used for ubiquitous or tissue specific expression. Further, virus-derived promoters, some of which are noted above, may be useful in the methods disclosed herein, e.g., CMV, HIV, adenovirus, and AAV promoters. In some aspects, the promoter is used together with at least one enhancer to increase the transcription efficiency. Non-limiting examples of enhancers include an interstitial retinoid-binding protein (IRBP) enhancer, an RSV enhancer or a CMV enhancer.

In some aspects, a promoter sequence can comprise, consist essentially of, or consist of a Rous sarcoma virus (RSV) LTR promoter sequence (optionally with the RSV enhancer), a cytomegalovirus (CMV) promoter sequence, an SV40 promoter sequence, a dihydrofolate reductase promoter sequence, a JeT promoter sequence, a strong a β-actin promoter sequence, a phosphoglycerol kinase (PGK) promoter sequence, a U6 promoter sequence, synapsin promoter, an H1 promoter sequence, a ubiquitous chicken β-actin hybrid (CBh) promoter sequence, a small nuclear RNA (U1a or U1b) promoter sequence, an MECP2 promoter sequence, an MeP418 promoter, an MeP426 promoter sequence, a small ubiquitous promoter sequence (also known as a Jet+I promoter sequence) MECP2 promoter sequence, a VMD2 promoter sequence, an mRho promoter sequence, an EFI promoter sequence, an EFla promoter sequence, a Ubc promoter sequence, a human β-actin promoter sequence, a CAG promoter sequence, a TRE promoter sequence, an Ac5 promoter sequence, a Polyhedrin promoter sequence, a CaMKIIa promoter sequence, a Gall promoter sequence, a TEF1 promoter sequence, a GDS promoter sequence, an ADH1 promoter sequence, a Ubi promoter sequence, a MeP426 promoter, or an α-1-antitrypsin (hAAT) promoter sequence.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) or synthetic techniques such that transcription of that gene is directed by the linked enhancer/promoter. Non-limiting examples of linked enhancer/promoter for use in the methods, compositions and constructs provided herein include a PDE promoter plus IRBP enhancer or a CMV enhancer plus U1a promoter. It is understood in the art that enhancers can operate from a distance and irrespective of their orientation relative to the location of an endogenous or heterologous promoter. It is thus further understood that an enhancer operating at a distance from a promoter is thus “operably linked” to that promoter irrespective of its location in the vector or its orientation relative to the location of the promoter.

As used throughout the disclosure, the term “operably linked” refers to the expression of a gene (i.e. a transgene) that is under the control of a promoter with which it is spatially connected. A promoter can be positioned 5′ (upstream) or 3′ (downstream) of a gene under its control. A promoter can be positioned 5′(upstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

In some aspects, a promoter sequence can comprise, consist essentially of, or consist of an MeP426 promoter sequence. A MeP426 promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO: 19.

In some aspects, a promoter sequence can comprise, consist essentially of, or consist of a JeT promoter sequence. A JeT promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO: 20.

In some aspects, a promoter sequence can comprise, consist essentially of, or consist of a Jet+I promoter sequence. A Jet+I promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO: 21.

In some aspects, a promoter sequence can comprise, consist essentially of, or consist of a MeP229 promoter sequence. A MeP229 promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO: 22.

In some aspects, a promoter sequence can comprise, consist essentially of, or consist of a hybrid chicken β-actin promoter sequence. A hybrid chicken β-actin promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO: 23.

As would be appreciated by the skilled artisan, a hybrid chicken β-actin promoter sequence can comprise a CMV sequence, a chicken β-actin promoter sequence, a chicken β-actin exon 1 sequence, a chicken β-actin intron 1 sequence, a minute virus of mice (MVM) intron sequence, or any combination thereof. In some aspects, a hybrid chicken β-actin promoter sequence can comprise, in the 5′ to 3′ direction, a CMV sequence, a chicken β-actin promoter sequence, chicken β-actin exon 1 sequence, a chicken β-actin intron 1 sequence and a minute virus of mice (MVM) intron sequence.

In some aspects, a CMV sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO: 28. The β-actin exon 1 sequence may comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO: 29. The chicken β-actin intron 1 sequence may comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO: 30. The MVM intron sequence may comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO: 31.

In some aspects, a promoter sequence can comprise, consist essentially of, or consist of a U6 promoter sequence. A U6 promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO: 224.

In some aspects, a promoter sequence can comprise, consist essentially of, or consist of a synapsin promoter sequence. A synapsin promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO: 25. A synapsin promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO: 26.

### Transgene Nucleic Acid Molecules

Transgene nucleic acid molecules can comprise, consist essentially of, or consist of any of the transgene nucleic acid molecules described above under the heading “isolated polynucleotides comprising transgene sequences”.

In some aspects, a transgene nucleic acid molecule present in an rAAV vector can be under transcriptional control of a promoter sequence also present in the same rAAV vector.

polyA Sequences

In some aspects, a polyadenylation (polyA) sequence can comprise any polyA sequence known in the art. The polyA sequence may be a synthetic polyA sequence or a polyA sequence derived from a naturally occurring protein. Non-limiting examples of polyA sequences include, but are not limited to, an MECP2 polyA sequence, a retinol dehydrogenase 1 (RDH1) polyA sequence, a bovine growth hormone (BGH) polyA sequence, an SV40 polyA sequence, a SPA49 polyA sequence, a sNRP-TK65 polyA sequence, a sNRP polyA sequence, or a TK65 polyA sequence.

Thus, a polyA sequence can comprise, consist essentially of, or consist of an MeCP2 polyA sequence, a retinol dehydrogenase 1 (RDH1) polyA sequence, a bovine growth hormone (BGH) polyA sequence, an SV40 polyA sequence, a SPA49 polyA sequence, a sNRP-TK65 polyA sequence, a sNRP polyA sequence, or a TK65 polyA sequence.

In some aspects, a polyA sequence can comprise, consist essentially of, or consist of an SV40 pA sequence. In some aspects, an SV40 pA sequence can comprise, consist essentially of, or consist of a nucleic acid sequence at least 65%, 70%, 75%, 80%, 85%, 90%, 95%, 96%, 97%, 98%, 99% or 100% (or any percentage in between) identical the sequence set forth in SEQ ID NO: 33.

In some aspects, a polyA sequence can comprise, consist essentially of, or consist of a BGH polyA sequence. In some aspects, an BGH polyA sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence set forth in SEQ ID NO: 34. In some aspects, an BGH polyA sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence set forth in SEQ ID NO: 35.

In some aspects, a polyA sequence be a synthetic polyA sequence. In some aspects, a synthetic polyA sequence can comprise, consist essentially of, or consist of a nucleic acid sequence at least 65%, 70%, 75%, 80%, 85%, 90%, 95%, 96%, 97%, 98%, 99% or 100% (or any percentage in between) identical the sequence set forth in SEQ ID NO: 36.

In certain embodiments, an rAAV vector disclosed herein comprises a Kozak sequence. In some aspects, an Kozak sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence set forth in SEQ ID NO: 32.

In certain embodiments, an rAAV vector disclosed herein comprises a Woodchuck Hepatitis Virus (WHV) Posttranscriptional Regulatory Element (WPRE). In some aspects, a WPRE sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence set forth in SEQ ID NO: 37.

In some aspects, an rAAV vector of the present disclosure can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NO: 38.

In some embodiments, an rAAV vector of the present disclosure consists of or comprises the sequence set forth in SEQ ID NO: 38 with one or more (e.g., 1, 2, 3, 4, 5, 6, 7, 8, 9, 10, 11, 12, 13, 14, 15, 16, 17, 18, 19, or 20) conservative amino acid substitutions.

In certain embodiments, an rAAV vector described herein comprises, in 5′ to 3′ order, a first AAV2 ITR of SEQ ID NO: 7; a Jet+I promoter of SEQ ID NO: 21; a codon optimized transgene encoding SLC13A5 of SEQ ID NO: 3; a synthetic polyA sequence of SEQ ID NO: 36; and a second AAV2 ITR of SEQ ID NO: 8.

### Bacterial Plasmids

In some aspects, the rAAV vectors of the present disclosure can be contained within a bacterial plasmid to allow for propagation of the rAAV vector in vitro. Thus, the present disclosure provides bacterial plasmids comprising any of the rAAV vectors described herein. A bacterial plasmid can further comprise an origin of replication sequence. A bacterial plasmid can further comprise an antibiotic resistance gene. A bacterial plasmid can further comprise a resistance gene promoter. A bacterial plasmid can further comprise a prokaryotic promoter. In some aspects, a bacterial plasmid of the present disclosure can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the nucleic acid sequence put forth in SEQ ID NO: 39.

### Origin of Replication Sequence

In some aspects, an origin of replication sequence can comprise, consist essentially of, or consist of any origin of replication sequence known in the art. The origin of replication sequence can be a bacterial origin of replication sequence, thereby allowing the rAAV vector comprising said bacterial origin of replication sequence to be produced, propagated and maintained in bacteria, using methods standard in the art.

### Antibiotic Resistance Genes

In some aspects, bacterial plasmids, rAAV vectors and/or rAAV viral vectors of the disclosure can comprise an antibiotic resistance gene.

In some aspects, an antibiotic resistance gene can comprise, consist essentially of, or consist of any antibiotic resistance genes known in the art. Examples of antibiotic resistance genes known in the art include, but are not limited to kanamycin resistance genes, spectinomycin resistance genes, streptomycin resistance genes, ampicillin resistance genes, carbenicillin resistance genes, bleomycin resistance genes, erythromycin resistance genes, polymyxin B resistance genes, tetracycline resistance genes and chloramphenicol resistance genes.

In some aspects, an antibiotic resistance gene can be a kanamycin resistance gene. In some aspects, a kanamycin resistance gene can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the nucleic acid sequence put forth in SEQ ID NO: 40.

### Resistance Gene Promoter

In some aspects, bacterial plasmids, rAAV vectors and/or rAAV viral vectors of the disclosure can comprise a resistance gene promoter. In some aspects, a resistance gene promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the nucleic acid sequence put forth in SEQ ID NO: 41.

### RepCap Sequences

In some aspects, bacterial plasmids, rAAV vectors and/or rAAV viral vectors of the disclosure can comprise a sequence encoding the rep proteins and capsid proteins of the rAAV (a “RepCap sequence”). In some embodiments, a RepCap sequence an comprise a nucleic acid encoding the rep and capsid proteins of AAV9. In some aspects, a RepCap sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the nucleic acid sequence put forth in SEQ ID NO: 42.

### AAV Viral Vectors

A “viral vector” is defined as a recombinantly produced virus or viral particle that contains a polynucleotide to be delivered into a host cell, either in vivo, ex vivo or in vitro. Examples of viral vectors include retroviral vectors, AAV vectors, lentiviral vectors, adenovirus vectors, alphavirus vectors and the like. Alphavirus vectors, such as Semliki Forest virus-based vectors and Sindbis virus-based vectors, have also been developed for use in gene therapy and immunotherapy. See, e.g., Schlesinger and Dubensky (1999) Curr. Opin. Biotechnol. 5:434-439 and Ying, et al. (1999) Nat. Med. 5(7):823-827.

An “AAV virion” or “AAV viral particle” or “AAV viral vector” or “rAAV viral vector” or “AAV vector particle” or “AAV particle” refers to a viral particle composed of at least one AAV capsid protein and an encapsidated polynucleotide rAAV vector. Thus, production of an rAAV viral vector necessarily includes production of an rAAV vector, as such a vector is contained within an rAAV vector.

As used herein, the term “viral capsid” or “capsid” refers to the proteinaceous shell or coat of a viral particle. Capsids function to encapsidate, protect, transport, and release into the host cell a viral genome. Capsids are generally comprised of oligomeric structural subunits of protein (“capsid proteins”). As used herein, the term “encapsidated” means enclosed within a viral capsid. The viral capsid of AAV is composed of a mixture of three viral capsid proteins: VP1, VP2, and VP3. The mixture of VP1, VP2 and VP3 contains 60 monomers that are arranged in a T=1 icosahedral symmetry in a ratio of 1:1:10 (VP1:VP2:VP3) or 1:1:20 (VP1:VP2:VP3) as described in Sonntag F et al., (June 2010). “A viral assembly factor promotes AAV2 capsid formation in the nucleolus”. Proceedings of the National Academy of Sciences of the United States of America. 107 (22): 10220-5, and Rabinowitz J E, Samulski R J (December 2000). “Building a better vector: the manipulation of AAV virions”. Virology. 278 (2): 301-8, each of which is incorporated herein by reference in its entirety.

The present disclosure provides an rAAV viral vector comprising: a) any of the rAAV vectors described herein, or complement thereof; and b) an AAV capsid protein.

The present disclosure provides an rAAV viral vector comprising: a) any of the rAAV vectors described herein; and b) an AAV capsid protein.

An AAV capsid protein can be any AAV capsid protein known in the art. An AAV capsid protein can be an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV 11 capsid protein, an AAV12 capsid protein, an AAV13 capsid protein, an AAVPHP.B capsid protein, an AAVrh74 capsid protein or an AAVrh.10 capsid protein.

### Alternative rAAV Vector and rAAV Viral Vector Embodiments

1. An rAAV vector, comprising, in the 5′ to 3′ direction
   a. a first AAV ITR sequence;
   b. a promoter sequence;
   c. a transgene nucleic acid molecule, wherein the transgene nucleic acid molecule comprises a nucleic acid sequence encoding for an SLC13A5 polypeptide;
   d. a polyA sequence; and
   e. a second AAV ITR sequence.
2. The rAAV vector of embodiment 1, wherein the SLC13A5 polypeptide comprises the amino acid sequence set forth in SEQ ID NO: 1 or SEQ ID NO: 2.
3. The rAAV vector of embodiment 2, wherein the SLC13A5 polypeptide comprises the amino acid sequence set forth in SEQ ID NO: 1.
4. The rAAV vector of embodiment 2, wherein the SLC13A5 polypeptide comprises the amino acid sequence set forth in SEQ ID NO: 2.
5. The rAAV vector of any one of the preceding embodiments, wherein the nucleic acid sequence encoding for an SLC13A5 polypeptide comprises the nucleic acid sequence set forth in any one of SEQ ID NOs: 3-6.
6. The rAAV vector of any one of the preceding embodiments, wherein the nucleic acid sequence encoding for an SLC13A5 polypeptide comprises the nucleic acid sequence set forth in SEQ ID NO: 3.
7. The rAAV vector of any one of the preceding embodiments, wherein the nucleic acid sequence encoding for an SLC13A5 polypeptide comprises the nucleic acid sequence set forth in SEQ ID NO: 4.
8. The rAAV vector of any one of the preceding embodiments, wherein the nucleic acid sequence encoding for an SLC13A5 polypeptide comprises the nucleic acid sequence set forth in SEQ ID NO: 5.
9. The rAAV vector of any one of the preceding embodiments, wherein the nucleic acid sequence encoding for an SLC13A5 polypeptide comprises the nucleic acid sequence set forth in SEQ ID NO: 6.
10. The rAAV vector of any one of the preceding embodiments, wherein the first AAV ITR sequence comprises the nucleic acid sequence set forth in any one of SEQ ID NOs: 7, 9, 11-14, 16 and 17.
11. The rAAV vector of any one of the preceding embodiments, wherein the first AAV ITR sequence comprises the nucleic acid sequence set forth in SEQ ID NO: 7.
12. The rAAV vector of any one of the preceding embodiments, wherein the second AAV ITR sequence comprises the nucleic acid sequence set forth in any one of SEQ ID NOs: 8, 10, 15, or 18.
13. The rAAV vector of any one of the preceding embodiments, wherein the second AAV ITR sequence comprises the nucleic acid sequence set forth in SEQ ID NO: 8.
14. The rAAV vector of any one of the preceding embodiments, wherein the promoter sequence comprises the nucleic acid sequence set forth in any one of SEQ ID NOs: 19-27.
15. The rAAV vector of any one of the preceding embodiments, wherein the promoter sequence comprises a synapsin promoter sequence.
16. The rAAV vector of any one of the preceding embodiments, wherein the synapsin promoter sequence comprises the nucleic acid sequence set forth in SEQ ID NO: 25.
17. The rAAV vector of any one of the preceding embodiments, wherein the synapsin promoter sequence comprises the nucleic acid sequence set forth in SEQ ID NO: 26.
18. The rAAV vector of any one of the preceding embodiments, wherein the promoter sequence comprises a JetI promoter sequence.
19. The rAAV vector of any one of the preceding embodiments, wherein the JetI promoter sequence comprises the nucleic acid sequence set forth in SEQ ID NO: 20.
20. The rAAV vector of any one of the preceding embodiments, wherein the polyA sequence comprises the nucleic acid sequence set forth in any one of SEQ ID NOs: 33-36.
21. The rAAV vector of any one of the preceding embodiments, wherein the polyA sequence comprises a BGH polyA sequence.
22. The rAAV vector of any one of the preceding embodiments, wherein the BGH polyA sequence comprises the nucleic acid sequence set forth in SEQ ID NO: 34.
23. The rAAV vector of any one of the preceding embodiments, wherein the BGH polyA sequence comprises the nucleic acid sequence set forth in SEQ ID NO: 35.
24. An rAAV vector of any one of the preceding embodiments, comprising, in the 5′ to 3′ direction
   a. a first AAV ITR sequence comprising the nucleic acid sequence of SEQ ID NO: 7;
   b. a promoter sequence comprising the nucleic acid sequence of SEQ ID NO: 21;
   c. a transgene nucleic acid molecule, wherein the transgene nucleic acid molecule comprises a nucleic acid sequence encoding for an SLC13A5 polypeptide, wherein the SLC13A5 polypeptide comprises the amino acid sequence of SEQ ID NO: 1 or SEQ ID NO: 2;
   d. a polyA sequence comprising the nucleic acid sequence of SEQ ID NO: 36; and
   e. a second AAV ITR sequence comprising the nucleic acid sequence of SEQ ID NO: 8.
25. An rAAV vector of any one of the preceding embodiments, wherein the rAAV vector comprises the nucleic acid sequence of SEQ ID NO: 38.
26. An rAAV viral vector comprising:
   a. an rAAV vector of any one of the preceding embodiments, or complement thereof; and
   b. an AAV capsid protein.
27. An rAAV viral vector comprising:
   a. an rAAV vector of any one of the preceding embodiments; and
   b. an AAV capsid protein.
28. The rAAV viral vector of any one of the preceding embodiments, wherein the AAV capsid protein is an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV 11 capsid protein, an AAV12 capsid protein, an AAV13 capsid protein, an AAVPHP.B capsid protein, an AAVrh74 capsid protein or an AAVrh.10 capsid protein.
29. The rAAV viral vector of embodiment 28, wherein the AAV capsid protein is an AAV1 capsid protein.
30. The rAAV viral vector of embodiment 28, wherein the AAV capsid protein is an AAV2 capsid protein.
31. The rAAV viral vector of embodiment 28, wherein the AAV capsid protein is an AAV3 capsid protein.
32. The rAAV viral vector of embodiment 28, wherein the AAV capsid protein is an AAV4 capsid protein.
33. The rAAV viral vector of embodiment 28, wherein the AAV capsid protein is an AAV5 capsid protein.
34. The rAAV viral vector of embodiment 28, wherein the AAV capsid protein is an AAV6 capsid protein.
35. The rAAV viral vector of embodiment 28, wherein the AAV capsid protein is an AAV7 capsid protein.
36. The rAAV viral vector of embodiment 28, wherein the AAV capsid protein is an AAV8 capsid protein.
37. The rAAV viral vector of embodiment 28, wherein the AAV capsid protein is an AAV9 capsid protein.
38. The rAAV viral vector of embodiment 28, wherein the AAV capsid protein is an AAV10 capsid protein.
39. The rAAV viral vector of embodiment 28, wherein the AAV capsid protein is an AAV 11 capsid protein.
40. The rAAV viral vector of embodiment 28, wherein the AAV capsid protein is an AAV12 capsid protein.
41. The rAAV viral vector of embodiment 28, wherein the AAV capsid protein is an AAV13 capsid protein.
42. The rAAV viral vector of embodiment 28, wherein the AAV capsid protein is an AAVPHP.B capsid protein.
43. The rAAV viral vector of embodiment 28, wherein the AAV capsid protein is an AAVrh74 capsid protein.
44. The rAAV viral vector of embodiment 28, wherein the AAV capsid protein is an AAVrh.10 capsid protein.

### Compositions and Pharmaceutical Compositions

The present disclosure provides compositions comprising any of the isolated polynucleotides, rAAV vectors, and/or rAAV viral vectors described herein. In some aspects, the compositions can be pharmaceutical compositions. Accordingly, the present disclosure provides pharmaceutical compositions comprising any of the isolated polynucleotides, rAAV vectors, and/or rAAV viral vectors described herein.

The pharmaceutical composition, as described herein, may be formulated by any methods known or developed in the art of pharmacology, which include but are not limited to contacting the active ingredients (e.g., viral particles or recombinant vectors) with an excipient and/or additive and/or other accessory ingredient, dividing or packaging the product to a dose unit. The viral particles of this disclosure may be formulated with desirable features, e.g., increased stability, increased cell transfection, sustained or delayed release, biodistributions or tropisms, modulated or enhanced translation of encoded protein in vivo, and the release profile of encoded protein in vivo.

As such, the pharmaceutical composition may further comprise saline, lipidoids, liposomes, lipid nanoparticles, polymers, lipoplexes, core-shell nanoparticles, peptides, proteins, cells transfected with viral vectors (e.g., for transplantation into a subject), nanoparticle mimics or combinations thereof. In some aspects, the pharmaceutical composition is formulated as a nanoparticle. In some aspects, the nanoparticle is a self-assembled nucleic acid nanoparticle.

A pharmaceutical composition in accordance with the present disclosure may be prepared, packaged, and/or sold in bulk, as a single unit dose, and/or as a plurality of single unit doses. The amount of the active ingredient is generally equal to the dosage of the active ingredient which would be administered to a subject and/or a convenient fraction of such a dosage such as, for example, one-half or one-third of such a dosage. The formulations of the invention can include one or more excipients and/or additives, each in an amount that together increases the stability of the viral vector, increases cell transfection or transduction by the viral vector, increases the expression of viral vector encoded protein, and/or alters the release profile of viral vector encoded proteins. In some aspects, the pharmaceutical composition comprises an excipient and/or additive. Non limiting examples of excipients and/or additives include solvents, dispersion media, diluents, or other liquid vehicles, dispersion or suspension aids, surface active agents, isotonic agents, thickening or emulsifying agents, preservatives, or combination thereof.

In some aspects, the pharmaceutical composition comprises a cryoprotectant. The term “cryoprotectant” refers to an agent capable of reducing or eliminating damage to a substance during freezing. Non-limiting examples of cryoprotectants include sucrose, trehalose, lactose, glycerol, dextrose, raffinose and/or mannitol.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

In some aspects, a pharmaceutical composition of the present disclosure can comprise phosphate-buffered saline (PBS), D-sorbitol or any combination thereof.

In some aspects, a pharmaceutical composition can comprise PBS, wherein the PBS is present at a concentration of about 100 mM to about 500 mM, or about 200 mM to about 400 mM, or about 300 mM to about 400 mM. In some aspects, the sodium chloride can be present at a concentration of about 350 mM.

In some aspects, a pharmaceutical composition can comprise D-sorbitol, wherein the D-sorbitol is present at a concentration of about 1% to about 10%, or about 2.5% to about 7.5%. In some aspects, the D-sorbitol can be present at a concentration of about 5%.

Thus, the present disclosure provides a pharmaceutical composition comprising an rAAV vector and/or rAAV viral vector of the present disclosure in a 350 mM phosphate-buffered saline solution comprising D-sorbitol at a concentration of 5%.

### Methods of Using the Compositions of the Disclosure

The present disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In one aspect, the subject is a mammal. Preferably, the subject is human.

This disclosure provides methods of preventing or treating a disease and/or disorder, comprising, consisting essentially of, or consisting of administering to a subject a therapeutically effective amount of any one of the rAAV vectors, rAAV viral vectors, compositions and/or pharmaceutical compositions disclosed herein.

In some aspects, the disease and/or disorder can be a genetic disorder involving the SLC13A5 gene. A genetic disorder involving the SLC13A5 gene can be SLC13A5 loss, misfunction and/or deficiency. Genetic disorders involving the SLC13A5 gene include, but are not limited to, epileptic encephalopathies, such as neonatal epileptic encephalopathy.

In some aspects, the disease can be a disorder involving the SLC13A5 protein. A genetic disorder involving the SLC13A5 protein can be SLC13A5 loss, misfunction and/or deficiency.

In some aspects, a disease can be a disease that is characterized by the loss-of-function of at least one copy of the SLC13A5 gene in the genome of a subject. In some aspects, a disease can be a disease that is characterized by a decrease in function of at least one copy of the SLC13A5 gene in the genome of a subject. In some aspects, a disease can be a disease that is characterized by at least one mutation in at least one mutation in at least one copy of the SLC13A5 gene in the genome of the subject.

A subject in the methods provided herein can be deficient in SLC13A5 and/or SLC13A5. As used herein, “SLC13A5 deficiency” means that a subject can have one or more mutations in the SLC13A5 gene or lacks a functional SLC13A5 gene. As used herein, “SLC13A5 deficiency” means that a subject can have one or more mutations in the SLC13A5 protein or lacks a functional SLC13A5 protein.

A mutation in an SLC13A5 gene or SLC13A5 protein can be any type of mutation that is known in the art. Non-limiting examples of mutations include somatic mutations, single nucleotide variants (SNVs), nonsense mutations, insertions, deletions, duplications, frameshift mutations, repeat expansions, short insertions and deletions (INDELs), long INDELs, alternative splicing, the products of alternative splicing, altered initiation of translation, the products of altered initiation of translation, proteomic cleavage, the products of proteomic cleavage.

In certain embodiments, a subject treated in accordance with a method described herein has a mutation in the SLC13A5 gene that is selected from the group consisting of c.103-1G>A, c.148T>C, c.231+2T>G, c.389G>A, c.425C>T, c.478G>T, c.511delG, c.644C>T, c.655G>A, c.680C>T, c.997C>T, c.1022G>A, c.1276-1 G>A, 1475T>C, 1514C>T, intronic CCDX 11079.1, a gene deletion of exons 2 to 4, and a gene deletion of exon 1 to 5. In some embodiments, a subject treated in accordance with a method described herein has a mutation in the SLC13A5 protein that is selected from the group consisting of C50R, G130D, T142M, Glu160*, E171Sfs*16, A215V, G219R, T227M, R333*, Trp341*, L492P, and P505L.

In some embodiments, the gene mutation is c.655G>A. In some embodiments, the gene mutations is c.680 C>T. In some embodiments, the protein mutations is G219R. In some embodiments, the protein mutation is T227M

In some aspects, a disease can be a disease that is characterized by a decrease in expression of the SLC13A5 gene in a subject as compared to a control subject that does not have the disease. In some aspects, the decrease in expression can be at least about 10%, or at least about 20%, or at least about 30%, or at least about 40%, or at least about 50%, or at least about 60%, or at least about 70%, or at least about 80%, or at least about 90%, or at least about 95%, or at least about 99%, or at least about 100%.

In some aspects, a disease can be a disease that is characterized by a decrease in the amount of SLC13A5 protein in a subject as compared to a control subject that does not have the disease. In some aspects, the decrease in the amount of SLC13A5 protein can be at least about 10%, or at least about 20%, or at least about 30%, or at least about 40%, or at least about 50%, or at least about 60%, or at least about 70%, or at least about 80%, or at least about 90%, or at least about 95%, or at least about 99%, or at least about 100%.

In some aspects, a disease can be a disease that is characterized by a decrease in the activity of SLC13A5 protein in a subject as compared to a control subject that does not have the disease. In some aspects, the decrease in the activity of SLC13A5 protein can be at least about 10%, or at least about 20%, or at least about 30%, or at least about 40%, or at least about 50%, or at least about 60%, or at least about 70%, or at least about 80%, or at least about 90%, or at least about 95%, or at least about 99%, or at least about 100%.

Methods of treatment can alleviate one or more symptoms of a disease and/or disorder described herein. In an embodiment, delivery of compositions described herein can prevent or delay development of detectable symptoms, if administered to a subject carrying a mutation in the SLC13A5 gene before symptoms become detectable. Therefore, treatment can be therapeutic or prophylactic.

Therapy refers to inhibition or reversal of established symptoms or phenotype. Therapy can also mean delay of onset of symptoms or phenotype. Prophylaxis means inhibiting or preventing development of symptoms in subjects not already displaying overt symptoms. Subjects not displaying overt symptoms can be identified early in life as carrying a loss of function mutation in the SLC13A5 gene by appropriate genetic testing performed before 18 months, 12 months, or 6 months of age.

A subject to be treated using the methods, compositions, pharmaceutical compositions, rAAV vectors or rAAV viral vectors of the present disclosure can have any of the diseases and/or symptoms described herein.

In some aspects, a subject can be less than 0.5 years of age, or less than 1 year of age, or less than 1.5 years of age, or less than 2 years of age, or at less than 2.5 years of age, or less than 3 years of age, or less than 3.5 years of age, or less than 3.5 years of age, or less than 4 years of age, or less than 4.5 years of age, or less than 5 years of age, or less than 5.5 years of age, or less than 6 years of age, or less than 6.5 years of age, or less than 7 years of age, or less than 7.5 years of age, or less than 8 years of age, or less than 8.5 years of age, or less than 9 years of age, or less than 9.5 years of age, or less than 10 years of age. In some aspects the subject can be less than 11 years of age, less than 12 years of age, less than 13 years of age, less than 14 years of age, less than 15 years of age, less than 20 years of age, less than 30 years of age, less than 40 years of age, less than 50 years of age, less than 60 years of age, less than 70 years of age, less than 80 years of age, less than 90 years of age, less than 100 years of age, less than 110 years of age, or less than 120 years of age. In some aspects, a subject can be less than 0.5 years of age. In some aspects, a subject can be less than 4 years of age. In some aspects, a subject can be less than 10 years of age.

The methods of treatment and prevention disclosed herein may be combined with appropriate diagnostic techniques to identify and select patients for the therapy or prevention.

The disclosure provides methods of increasing the level of a protein in a host cell, comprising contacting the host cell with any one of the rAAV viral vectors disclosed herein, wherein the rAAV viral vectors comprises any one of the rAAV vectors disclosed herein, comprising a transgene nucleic acid molecule encoding the protein. In some aspects, the protein is a therapeutic protein. In some aspects, the host cell is in vitro, in vivo, or ex vivo. In some aspects, the host cell is derived from a subject. In some aspects, the subject suffers from a disorder, which results in a reduced level and/or functionality of the protein, as compared to the level and/or functionality of the protein in a normal subject.

In some aspects, the level of the protein is increased to level of about 1×10⁻⁷ ng, about 3×10⁻⁷ ng, about 5×10⁻⁷ ng, about 7×10⁻⁷ ng, about 9×10⁻⁷ ng, about 1×10⁻⁶ ng, about 2×10⁻⁶ ng, about 3×10⁻⁶ ng, about 4×10⁻⁶ ng, about 6×10⁻⁶ ng, about 7×10⁻⁶ ng, about 8×10⁻⁶ ng, about 9×10⁻⁶ ng, about 10×10⁻⁶ ng, about 12×10⁻⁶ ng, about 14×10⁻⁶ ng, about 16×10⁻⁶ ng, about 18×10⁻⁶ ng, about 20×10⁻⁶ ng, about 25×10⁻⁶ ng, about 30×10⁻⁶ ng, about 35×10⁻⁶ ng, about 40×10⁻⁶ ng, about 45×10⁻⁶ ng, about 50×10⁻⁶ ng, about 55×10⁻⁶ ng, about 60×10⁻⁶ ng, about 65×10⁻⁶ ng, about 70×10⁻⁶ ng, about 75×10⁻⁶ ng, about 80×10⁻⁶ ng, about 85×10⁻⁶ ng, about 90×10⁻⁶ ng, about 95×10⁻⁶ ng, about 10×10⁻⁵ ng, about 20×10⁻⁵ ng, about 30×10⁻⁵ ng, about 40×10⁻⁵ ng, about 50×10⁻⁵ ng, about 60×10⁻⁵ ng, about 70×10⁻⁵ ng, about 80×10⁻⁵ ng, or about 90×10⁻⁵ ng in the host cell.

The expression levels of a gene (e.g., SLC13A5) or a protein (e.g., SLC13A5) may be determined by any suitable method known in the art or described herein. Protein levels may be determined, for example, by Western Blotting, immunohistochemistry and flow cytometry. Gene expression may be determined, for example, by quantitative PCR, gene sequencing, and RNA sequencing.

The disclosure provides methods of introducing a gene of interest to a cell in a subject comprising contacting the cell with an effective amount of any one of the rAAV viral vectors disclosed herein, wherein the rAAV viral vectors contain any one of the rAAV vectors disclosed herein, comprising the gene of interest.

In some aspects of the methods of the present disclosure, a subject can also be administered a prophylactic immunosuppressant treatment regimen in addition to being administered an rAAV vector or rAAV viral vector of the present disclosure. In some aspects, an immunosuppressant treatment regimen can comprise administering at least one immunosuppressive therapeutic. Non limiting examples of immunosuppressive therapeutics include, but are not limited to, Sirolimus (rapamycin), acetaminophen, diphenhydramine, IV methylprednisolone, prednisone, or any combination thereof.

An immunosuppressive therapeutic can be administered prior to the day of administration of the rAAV vector and/or rAAV viral vector, on the same day as the administration of the rAAV vector and/or rAAV viral vector, or any day following the administration of the rAAV vector and/or rAAV viral vector.

A “subject” of diagnosis or treatment is a cell or an animal such as a mammal, or a human. The terms “subject” and “patient” are used interchangeably herein. A subject is not limited to a specific species and includes non-human animals subject to diagnosis or treatment and those subject to infections or animal models, including, without limitation, simian, murine, rat, canine, or leporid species, as well as other livestock, sport animals, or pets. In some aspects, the subject is a human. In some embodiments, the subject is a human child, e.g., a child of less than five years of age. In some embodiments, the subject is a human newborn, e.g., a newborn of less than one month, less than two months, less than three months, or less than four months of age.

As used herein, “treating” or “treatment” of a disease in a subject refers to (1) inhibiting the disease or arresting its development; or (2) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

As used herein, “preventing” or “prevention” of a disease refers to preventing the symptoms or disease from occurring in a subject that is predisposed or does not yet display symptoms of the disease.

As used herein the term “effective amount” intends to mean a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of gene therapy, the effective amount can be the amount sufficient to result in regaining part or full function of a gene that is deficient in a subject. In some aspects, the effective amount of an rAAV viral vector is the amount sufficient to result in expression of a gene in a subject such that an SLC13A5 polypeptide is produced. In some aspects, the effective amount is the amount required to decrease the frequency of seizures in subject by at least 10%, at least 20%, at least 30%, at least 40%, at least 50%, at least 60%, at least 70%, at least 80%, or at least 90% compared to a subject who has not been administered an rAAV viral vector described herein or has been administered a control treatment. The skilled artisan will be able to determine appropriate amounts depending on these and other factors.

In some aspects, the effective amount will depend on the size and nature of the application in question. It will also depend on the nature and sensitivity of the target subject and the methods in use.

The skilled artisan will be able to determine the effective amount based on these and other considerations. The effective amount may comprise, consist essentially of, or consist of one or more administrations of a composition depending on the embodiment.

As used herein, the term “administer” or “administration” intends to mean delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and other animals, treating veterinarian.

Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. It is noted that dosage may be impacted by the route of administration. Suitable dosage formulations and methods of administering the agents are known in the art. Non-limiting examples of such suitable dosages may be as low as 10⁹ vector genomes to as much as 10¹⁷ vector genomes per administration.

In some aspects of the methods described herein, the number of viral particles (e.g., rAAV viral vectors) administered to the subject ranges from about 10⁹ to about 10¹⁷. In some aspects, about 10¹⁰ to about 10¹², about 10¹¹ to about 10¹³, about 10¹¹ to about 10¹², about 10¹¹ to about 10¹⁴, about 10¹² to about 10¹⁶, about 10¹³ to about 10¹⁶, about 10¹⁴ to about 10¹⁵, about 5×10¹¹ to about 5×10¹², about 10¹¹ to about 10¹⁸, about 10¹³ to about 10¹⁶, or about 10¹² to about 10¹³ viral particles are administered to the subject.

In some aspects of the methods described herein, the number of viral particles (e.g., rAAV viral vectors) administered to the subject is at least about 10¹⁰, or at least about 10¹¹, or at least about 10¹², or at least about 10¹³, or at least about 10¹⁴, or at least about 10¹⁵, or at least about 10¹⁶, or at least about 10¹⁷ viral particles.

In some aspects of the methods described herein, the number of vector genomes (e.g., rAAV viral vectors) administered to the subject ranges from about 10⁹ to about 10¹⁷. In some aspects, about 10¹⁰ to about 10¹², about 10¹¹ to about 10¹³, about 10¹¹ to about 10¹², about 10¹¹ to about 10¹⁴, about 10¹² to about 10¹⁶, about 10¹³ to about 10¹⁶, about 10¹⁴ to about 10¹⁵, about 5×10¹¹ to about 5×10¹², about 10¹¹ to about 10¹⁸, about 10¹³ to about 10¹⁶, or about 10¹² to about 10¹³ vector genomes are administered to the subject.

In some aspects of the methods described herein, the number of vector genomes (e.g., rAAV viral vectors) administered to the subject is at least about 10¹⁰, or at least about 10¹¹, or at least about 10¹², or at least about 10¹³, or at least about 10¹⁴, or at least about 10¹⁵, or at least about 10¹⁶, or at least about 10¹⁷ vector genomes. In some aspects, 2×10¹¹ or about 8×10¹¹ vector genomes are administered to the subject.

In some aspects of the methods described herein, the number of viral particles (e.g., rAAV viral vectors) administered to the subject can depend on the age of the subject. In non-limiting examples, a subject that is 7 years of age or older can be administered about 10×10¹⁴ viral particles, a subject that is about 4 years of age to about 7 years of age can be administered about 10×10¹⁴ viral particles, a subject that is about 3 years of age to about 4 years of age can be administered about 9×10¹⁴ viral particles, a subject that is about 2 years of age to about 3 years of age can be about 8.2×10¹⁴ viral particles, a subject that is about 1 year of age to about 2 years of age can be administered about 7.3×10¹⁴ viral particles, a subject that is about 0.5 years of age to about 1 year of age can be administered about 4×10¹⁴ viral particles, or a subject that is less than 0.5 years of age can be administered 3×10¹⁴ viral particles.

In some aspects, the amounts of viral particles in a composition, pharmaceutical composition, or the amount of viral particles administered to a patient can calculated based on the percentage of viral particles that are predicted to contain viral genomes.

In some aspects, rAAV viral vectors of the present disclosure can be introduced to the subject intravenously, intrathecally (IT), intracisterna-magna (ICM) intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally; such introduction may also be intra-arterial, intracardiac, subventricular, epidural, intracerebral, intracerebroventricular, sub-retinal, intravitreal, intraarticular, intraperitoneal, intrauterine, intranerve or any combination thereof. In some aspects, the viral particles are delivered to a desired target tissue, e.g., to the lung, eye, or CNS, as non-limiting examples. In some aspects, delivery of viral particles is systemic. The intracisternal route of administration involves administration of a drug directly into the cerebrospinal fluid of the brain ventricles. It could be performed by direct injection into the cisterna magna or via a permanently positioned tube. In some aspects, the rAAV viral vectors of the present disclosure are administered intrathecally (IT). In some aspects, the rAAV viral vectors of the present disclosure are administered intracisterna-manga (ICM).

In some aspects, the rAAV viral vectors of the present disclosure repair a gene deficiency in a subject. In some aspects, the ratio of repaired target polynucleotide or polypeptide to unrepaired target polynucleotide or polypeptide in a successfully treated cell, tissue, organ or subject is at least about 1.5:1, about 2:1, about 3:1, about 4:1, about 5:1, about 6:1, about 7:1, about 8:1, about 9:1, about 10:1, about 20:1, about 50:1, about 100:1, about 1000:1, about 10,000:1, about 100,000:1, or about 1,000,000:1. The amount or ratio of repaired target polynucleotide or polypeptide can be determined by any method known in the art, including but not limited to western blot, northern blot, Southern blot, PCR, sequencing, mass spectrometry, flow cytometry, immunohistochemistry, immunofluorescence, fluorescence in situ hybridization, next generation sequencing, immunoblot, and ELISA.

Administration of the rAAV vectors, rAAV viral vectors, compositions or pharmaceutical compositions of this disclosure can be effected in one dose, continuously or intermittently throughout the course of treatment. In some aspects, the rAAV vectors, rAAV viral vectors, compositions, or pharmaceutical compositions of this disclosure are parenterally administered by injection, infusion, or implantation.

In some aspects, the rAAV viral vectors of this disclosure show enhanced tropism for brain and cervical spine. In some aspects, the rAAV viral vectors of the disclosure can cross the blood-brain-barrier (BBB).

In some embodiments, the subject is administered one single dose of a recombinant rAAV provided herein in its lifetime. In some embodiments, the subject is administered repeat doses of the recombinant rAAV provided herein. These repeat doses may contain the same amount of rAAV particles or they may contain different amounts of rAAV particles. In some embodiments, the subject is administered repeat doses of the rAAV about every 6 months, about every 9 months, about every 12 months, about every 15 months, about every 18 months, about every 2 years, about every 3 years, about every 4 years, about every 5 years, about every 6 years, about every 7 years, about every 8 years, about every 9 years, or about every 10 years.

### Methods of Manufacture

A variety of approaches may be used to produce rAAV viral vectors of the present disclosure.

In some aspects, packaging is achieved by using a helper virus or helper plasmid and a cell line. The helper virus or helper plasmid contains elements and sequences that facilitate viral vector production. In another aspect, the helper plasmid is stably incorporated into the genome of a packaging cell line, such that the packaging cell line does not require additional transfection with a helper plasmid.

In some aspects, the cell is a packaging or helper cell line. In some aspects, the helper cell line is eukaryotic cell; for example, an HEK 293 cell or 293T cell. In some aspects, the helper cell is a yeast cell or an insect cell.

In some aspects, the cell comprises a nucleic acid encoding a tetracycline activator protein; and a promoter that regulates expression of the tetracycline activator protein. In some aspects, the promoter that regulates expression of the tetracycline activator protein is a constitutive promoter. In some aspects, the promoter is a phosphoglycerate kinase promoter (PGK) or a CMV promoter.

A helper plasmid may comprise, for example, at least one viral helper DNA sequence derived from a replication-incompetent viral genome encoding in trans all virion proteins required to package a replication incompetent AAV, and for producing virion proteins capable of packaging the replication-incompetent AAV at high titer, without the production of replication-competent AAV.

Helper plasmids for packaging AAV are known in the art, see, e.g., U.S. Patent Pub. No. 2004/0235174 A1, incorporated herein by reference. As stated therein, an AAV helper plasmid may contain as helper virus DNA sequences, by way of non-limiting example, the Ad5 genes E2A, E4 and VA, controlled by their respective original promoters or by heterologous promoters. AAV helper plasmids may additionally contain an expression cassette for the expression of a marker protein such as a fluorescent protein to permit the simple detection of transfection of a desired target cell.

The disclosure provides methods of producing rAAV viral vectors comprising transfecting a packaging cell line with any one of the AAV helper plasmids disclosed herein; and any one of the rAAV vectors disclosed herein. In some aspects, the AAV helper plasmid and rAAV vector are co-transfected into the packaging cell line. In some aspects, the cell line is a mammalian cell line, for example, human embryonic kidney (HEK) 293 cell line. The disclosure provides cells comprising any one of the rAAV vectors and/or rAAV viral vectors disclosed herein.

As used herein, the term “helper” in reference to a virus or plasmid refers to a virus or plasmid used to provide the additional components necessary for replication and packaging of any one of the rAAV vectors disclosed herein. The components encoded by a helper virus may include any genes required for virion assembly, encapsidation, genome replication, and/or packaging. For example, the helper virus or plasmid may encode necessary enzymes for the replication of the viral genome. Non-limiting examples of helper viruses and plasmids suitable for use with AAV constructs include pHELP (plasmid), adenovirus (virus), or herpesvirus (virus). In some aspects, the pHELP plasmid may be the pHELPK plasmid, wherein the ampicillin expression cassette is exchanged with a kanamycin expression cassette.

As used herein, a packaging cell (or a helper cell) is a cell used to produce viral vectors.

Producing recombinant AAV viral vectors requires Rep and Cap proteins provided in trans as well as gene sequences from Adenovirus that help AAV replicate. In some aspects, Packaging/helper cells contain a plasmid is stably incorporated into the genome of the cell. In other aspects, the packaging cell may be transiently transfected. Typically, a packaging cell is a eukaryotic cell, such as a mammalian cell or an insect cell.

### Kits

The isolated polynucleotides, rAAV vectors, rAAV viral vectors, compositions, and/or pharmaceutical compositions described herein may be assembled into pharmaceutical or diagnostic or research kits to facilitate their use in therapeutic, diagnostic, or research applications. In some aspects, the kits of the present disclosure include any one of the isolated polynucleotides, rAAV vectors, rAAV viral vectors, compositions, pharmaceutical compositions, host cells, isolated tissues, as described herein.

In some aspects, a kit further comprises instructions for use. Specifically, such kits may include one or more agents described herein, along with instructions describing the intended application and the proper use of these agents. In some aspects, the kit may include instructions for mixing one or more components of the kit and/or isolating and mixing a sample and applying to a subject. In some aspects, agents in a kit are in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. In some aspects, the compositions may be provided in a preservation solution (e.g., cryopreservation solution). Non-limiting examples of preservation solutions include DMSO, paraformaldehyde, and CryoStor® (Stem Cell Technologies, Vancouver, Canada). In some aspects, the preservation solution contains an amount of metalloprotease inhibitors.

In some aspects, the kit contains any one or more of the components described herein in one or more containers. Thus, in some aspects, the kit may include a container housing agents described herein. The agents may be in the form of a liquid, gel or solid (powder). The agents may be prepared sterilely, packaged in a syringe and shipped refrigerated. Alternatively, they may be housed in a vial or other container for storage. A second container may have other agents prepared sterilely.

Alternatively, the kit may include the active agents premixed and shipped in a syringe, vial, tube, or other container. The kit may have one or more or all of the components required to administer the agents to a subject, such as a syringe, topical application devices, or IV needle tubing and bag.

### Further Definitions

Unless the context indicates otherwise, it is specifically intended that the various features of the invention described herein can be used in any combination. Moreover, the disclosure also contemplates that, in some aspects, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified aspects, embodiments, features, and terms intend to include both the recited aspect, embodiment, feature, or term and biological equivalents thereof.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (RI. Freshney, ed. (1987)).

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps and those that do not materially affect the basic and novel characteristic(s) of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure. In each instance herein any of the terms “comprising,” “consisting essentially of,” and “consisting of” can be replaced with either of the other two terms, while retaining their ordinary meanings. Any single term, single element, single phrase, group of terms, group of phrases, or group of elements described herein can each be specifically excluded from the claims.

All numerical designations, e.g., pH, temperature, time, concentration, and molecular weight, including ranges, are approximations which are varied (+) or (−) by increments of 1.0 or 0.1, as appropriate, or, alternatively, by a variation of +/−15%, 10%, 5%, 2%. It is to be understood, although not always explicitly stated, that all numerical designations are preceded by the term “about”. It also is to be understood, although not always explicitly stated, that the reagents described herein are merely exemplary and that equivalents of such are known in the art. The term “about,” as used herein when referring to a measurable value such as an amount or concentration and the like, is meant to encompass variations of 20%, 10%, 5%, 1%, 0.5%, or even 0.1% of the specified amount.

The terms “acceptable,” “effective,” or “sufficient” when used to describe the selection of any components, ranges, dose forms, etc. disclosed herein intend that said component, range, dose form, etc. is suitable for the disclosed purpose.

Also, as used herein, “and/or” refers to and encompasses any and all possible combinations of one or more of the associated listed items, as well as the lack of combinations when interpreted in the alternative (“or”).

Unless specifically recited, the term “host cell” includes a eukaryotic host cell, including, for example, fungal cells, yeast cells, higher plant cells, insect cells and mammalian cells. Non-limiting examples of eukaryotic host cells include simian, bovine, porcine, murine, rat, avian, reptilian and human, e.g., HEK293 cells and 293T cells.

The term “isolated” as used herein refers to molecules or biologicals or cellular materials being substantially free from other materials.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising, consisting essentially of, or consisting of purine and pyrimidine bases or other natural, chemically or biochemically modified, non-natural, or derivatized nucleotide bases.

A “gene” refers to a polynucleotide containing at least one open reading frame (ORF) that is capable of encoding a particular polypeptide or protein. A “gene product” or, alternatively, a “gene expression product” refers to the amino acid sequence (e.g., peptide or polypeptide) generated when a gene is transcribed and translated.

As used herein, “expression” refers to the two-step process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell.

“Under transcriptional control” is a term well understood in the art and indicates that transcription of a polynucleotide sequence, usually a DNA sequence, depends on its being operatively linked to an element that contributes to the initiation of, or promotes, transcription. “Operatively linked” intends that the polynucleotides are arranged in a manner that allows them to function in a cell. In one aspect, promoters can be operatively linked to the downstream sequences.

The term “encode” as it is applied to polynucleotides and/or nucleic acid sequences refers to a polynucleotide and/or nucleic acid sequence which is said to “encode” a polypeptide if its base sequence is identical to the base sequence of the RNA transcript (e.g. mRNA transcript) that is translated into the polypeptide and/or a fragment thereof. The antisense strand is the complement of such a nucleic acid, and the encoding sequence can be deduced therefrom.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise, consist essentially of, or consist of a protein's or peptide's sequence. As used herein the term “amino acid” refers to either natural and/or unnatural or synthetic amino acids, including glycine and both the D and L optical isomers, amino acid analogs and peptidomimetics.

As used herein, the term “signal peptide” or “signal polypeptide” intends an amino acid sequence usually present at the N-terminal end of newly synthesized secretory or membrane polypeptides or proteins. It acts to direct the polypeptide to a specific cellular location, e.g. across a cell membrane, into a cell membrane, or into the nucleus. In some aspects, the signal peptide is removed following localization. Examples of signal peptides are well known in the art. Non-limiting examples are those described in U.S. Pat. Nos. 8,853,381, 5,958,736, and 8,795,965. In some aspects, the signal peptide can be an IDUA signal peptide.

The terms “equivalent” or “biological equivalent” are used interchangeably when referring to a particular molecule, biological material, or cellular material and intend those having minimal homology while still maintaining desired structure or functionality. Non-limiting examples of equivalent polypeptides include a polypeptide having at least about 60%, at least about 65%, at least about 70%, at least about 75%, at least about 80%, at least about 85%, at least about 90%, at least about 95% identity or at least about 99% identity to a reference polypeptide (for instance, a wild-type polypeptide); or a polypeptide which is encoded by a polynucleotide having at least about 70%, at least about 75%, at least about 80%, at least about 85%, at least about 90%, at least about 95% identity, at least about 97% sequence identity or at least about 99% sequence identity to the reference polynucleotide (for instance, a wild-type polynucleotide).

“Homology” or “identity” or “similarity” refers to sequence similarity between two peptides or between two nucleic acid molecules. Percent identity can be determined by comparing a position in each sequence that may be aligned for purposes of comparison. When a position in the compared sequence is occupied by the same base or amino acid, then the molecules are identical at that position. A degree of identity between sequences is a function of the number of matching positions shared by the sequences. “Unrelated” or “non-homologous” sequences share less than 40% identity, less than 25% identity, with one of the sequences of the present disclosure. Alignment and percent sequence identity may be determined for the nucleic acid or amino acid sequences provided herein by importing said nucleic acid or amino acid sequences into and using ClustalW (available at https://genome.jp/tools-bin/clustalw/). For example, the ClustalW parameters used for performing the protein sequence alignments found herein were generated using the Gonnet (for protein) weight matrix. In some aspects, the ClustalW parameters used for performing nucleic acid sequence alignments using the nucleic acid sequences found herein are generated using the ClustalW (for DNA) weight matrix.

As used herein, amino acid modifications may be amino acid substitutions, amino acid deletions or amino acid insertions. Amino acid substitutions may be conservative amino acid substitutions or non-conservative amino acid substitutions. A conservative replacement (also called a conservative mutation, a conservative substitution or a conservative variation) is an amino acid replacement in a protein that changes a given amino acid to a different amino acid with similar biochemical properties (e.g., charge, hydrophobicity or size). As used herein, “conservative variations” refer to the replacement of an amino acid residue by another, biologically similar residue. Examples of conservative variations include the substitution of one hydrophobic residue such as isoleucine, valine, leucine or methionine for another; or the substitution of one charged or polar residue for another, such as the substitution of arginine for lysine, glutamic acid for aspartic acid, glutamine for asparagine, and the like. Other illustrative examples of conservative substitutions include the changes of: alanine to serine; asparagine to glutamine or histidine; aspartate to glutamate; cysteine to serine; glycine to proline; histidine to asparagine or glutamine; lysine to arginine, glutamine, or glutamate; phenylalanine to tyrosine, serine to threonine; threonine to serine; tryptophan to tyrosine; tyrosine to tryptophan or phenylalanine; and the like.

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is a DNA molecule that is typically separate from and capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or, alternatively, the proteins produced may act as toxins under similar circumstances. It is known in the art that while plasmid vectors often exist as extrachromosomal circular DNA molecules, plasmid vectors may also be designed to be stably integrated into a host chromosome either randomly or in a targeted manner, and such integration may be accomplished using either a circular plasmid or a plasmid that has been linearized prior to introduction into the host cell.

“Plasmids” used in genetic engineering are called “plasmid vectors”. Many plasmids are commercially available for such uses. The gene to be replicated is inserted into copies of a plasmid containing genes that make cells resistant to particular antibiotics, and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria or eukaryotic cells containing a plasmid harboring the gene of interest, which can be induced to produce large amounts of proteins from the inserted gene.

In aspects where gene transfer is mediated by a DNA viral vector, such as an adenovirus (Ad) or adeno-associated virus (AAV), a vector construct refers to the polynucleotide comprising, consisting essentially of, or consisting of the viral genome or part thereof, and a transgene.

The term “tissue” is used herein to refer to tissue of a living or deceased organism or any tissue derived from or designed to mimic a living or deceased organism. The tissue may be healthy, diseased, and/or have genetic mutations. The biological tissue may include any single tissue (e.g., a collection of cells that may be interconnected), or a group of tissues making up an organ or part or region of the body of an organism. The tissue may comprise, consist essentially of, or consist of a homogeneous cellular material or it may be a composite structure such as that found in regions of the body including the thorax which for instance can include lung tissue, skeletal tissue, and/or muscle tissue. Exemplary tissues include, but are not limited to those derived from liver, lung, thyroid, skin, pancreas, blood vessels, bladder, kidneys, brain, biliary tree, duodenum, abdominal aorta, iliac vein, heart and intestines, including any combination thereof.

### EXAMPLES

### Example 1: Study to Evaluate the Safety of AAV9/hSLC13A5 when Administered Intravenously in 8-Week Old Wild-Type C57BL/6 Mice

This study was designed to characterize the toxicity of AAV9/hSLC13A5, which comprises SEQ ID NO: 38. In an earlier study, IV delivery of 3.2×10¹² vg AAV9/hSLC13A5 per animal (neonate) was found to be toxic in 60% of treated animals, and all animals that received greater than 2.2×10¹⁵ vg/kg died. The cause of toxicity is unknown; however, deaths are thought to have resulted from the high number of viral particles received in neonates. This was further supported by the lack of long-term toxicity in surviving AAV9/hSLC13A5 treated mice that displayed robust SLC13A5 protein expression in the brain. In this study, to further characterize the toxicity of AAV9/hSLC13A5, a standard IV dose of 1×10¹⁴ vg/kg AAV9/hSLC13A5 was delivered in juvenile WT C57BL/6J mice with evaluation of the animals over a 15 month period. AAV9/hSLC13A5 comprises AAV9 capsids that are packaged with the self-complementary AAV genome comprising a mutant AAV2 inverted terminal repeat (ITR) with the D element deleted, the “UsP” promoter, codon-optimized human SLC13A5 DNA coding sequence, the polyadenylation signal, and wildtype AAV2 ITR.

### Methods

Wildtype Male and Female C57BL/6J littermates were either left untreated as controls (n=14, 7 females, 7 males) or received a tail vein injection of 1×10¹⁴ vg/kg of AAV9/hSLC13A5 (n=14, 7 females, 7 males) at 7-8 weeks of age. Animals were then observed and weighed 3 times per week for 8 weeks post-dosing. At 8 weeks post-dosing, a small group of animals were necropsied for an interim analysis. Remaining animals were necropsied at 15 months post-dosing for histological analysis. At time points beyond 8 weeks post-doing animals continued to be weighed and observed one time per week until 9 months post-injection and then monthly thereafter.

Treated mice received a tail vein IV injection of 1×10¹⁴ vg/kg AAV9/hSLC13A5 (Table 1). The volume was dependent upon the mouse's weight and the volume range for IV injected animals was 98-116 μL (104.41±2.32 μL) for females and 111-135 μL (124±2.85 μL) for males.

TABLE 1
Experimental Design

| | | | | | | Terminal Endpoint | |
|---|---|---|---|---|---|---|---|
| | | | Body Weight at | | | (Post-Injection)^{c} | |
| | | | Injection (g)^{b} | | Age at | Interim | Study End |
| | | Dose | Male | Females | Dosing | (n = 4 | (n = 10 |
| Group | Route^{a} | (vg/kg) | (n = 7) | (n = 7) | (weeks) | per group) | per group) |
| 1 | Untreated | — | 20.2 ± | 16.6 ± | 7-8 | 8 weeks | 15 months |
| | | | 0.82 | 0.45 | | | |
| 2 | IV | 1 × 10¹⁴ | 20.0 ± | 16.7 ± | 7-8 | 8 weeks | 15 months |
| | | | 0.48 | 0.48 | | | |

^{a}IV: intravenously injected through the tail vein.
^{b}Mean ± SEM
^{c}Mice were sacrificed at the indicated time post-injection for histopathology and compared to age- and sex-matched mice.

Mice were monitored for clinical signs, adverse events, and mortality following the treatment every week. Mice were weighed 3 times per week for the first 8 weeks, then one time per week until 9 months post-injection and then monthly thereafter. Animals that had lost weight from the previous time point were further observed for motor deficits and malocclusion.

At 8 weeks post-injection, blood was collected from a total of 8 mice (n=2/sex/group) for interim analysis. At the study-endpoint (15 months post-dosing), blood from the remaining 20 animals, (n=5/sex/group), was collected. Blood was collected as a terminal heart draw. Serum from 8 weeks post-dosing were analyzed for blood biochemistry, including total bilirubin (TBIL), albumin (ALB), aspartate aminotransferase (AST), blood urea nitrogen (BUN), and creatine kinase (CK). Levels of these biochemical variables indicate the function of kidney and liver.

Terminal tissue samples were collected for histopathological or clinical chemistry assessment 8 weeks or 15 months following treatment.

On the day of necropsy for the interim analysis group, animals (n=8) were weighed and then deeply anesthetized with an overdose of avertin (0.04 mL/g of a 1.25% solution). Blood was collected as a terminal heart draw. Animals were then perfused with cold PBS containing 1 μg/mL heparin. Tissues were quickly collected, divided and one portion flash frozen in liquid nitrogen and the other portion drop-fixed in 10% neutralized-buffered formalin (NBF) for histological analysis. For the interim group, the following tissues were analyzed: brain, liver, heart, and kidney.

On the day of necropsy for the end-point group, animals (n=20) were weighed and then deeply anesthetized as above. Blood was collected as a terminal heart draw. Animals were then perfused with cold PBS containing 1 μg/mL heparin followed by perfusion with 10% NBF for tissue collection. For the end-point group, brain, heart, skeletal muscle, liver, lung, gonad, spleen, kidney, sciatic nerve, cervical, thoracic, and lumbar spinal cord were analyzed for all animals if collected.

Student's unpaired t-test was used to analyze blood data. Body weight data were analyzed using Repeat ANOVA, with factors treatment or days-post injection, and followed by with Sidak's multiple comparison test. For all comparisons, statistical significance was set at p<0.05. Data were analyzed and graphed using GraphPad Prism software (v. 9.1.0).

### Results

There were no early deaths before the two planned endpoints (8 weeks post-dosing for interim analysis and 15 months post-dosing for endpoint analysis) (FIG. 1). There were no outward signs of toxicity noted over the duration of the study.

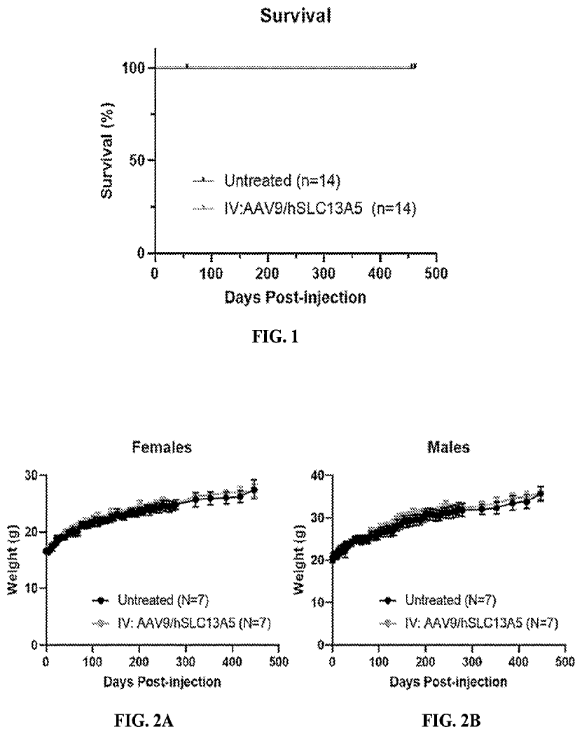

Body weight was monitored to assess the overall health of the animals. There was no significant difference of body weight between groups within male or female mice at any points of assessment (FIGS. 2A and 2B), demonstrating that the IV delivered dose of 1×10¹⁴ vg/kg was well tolerated in the wildtype C57BL/6J mice up to 15 months following treatment.

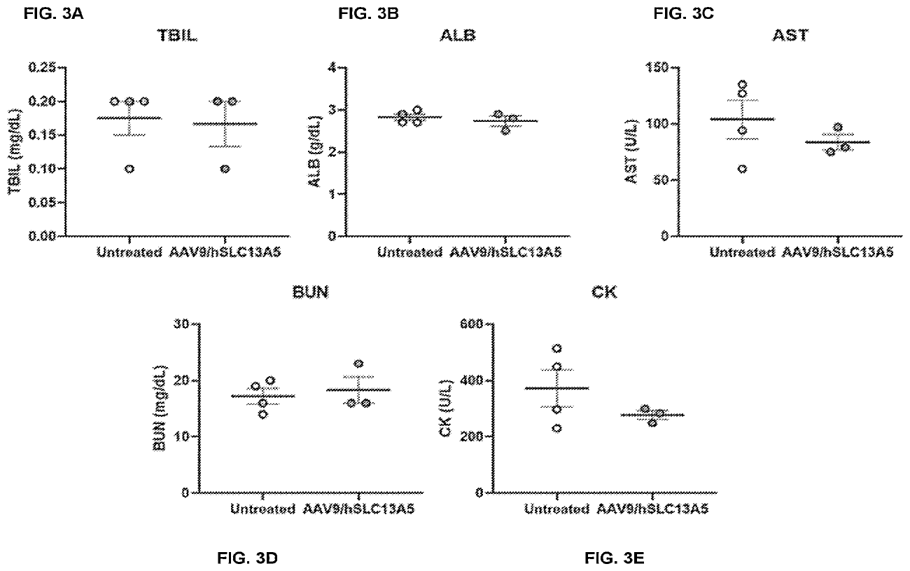

Blood biochemistry analysis from serum of the 8-weeks post-dosing interim analysis group did not show significant changes post-treatment (FIGS. 3A-3E). Only one serum sample from the end-point study group (15 months post-dosing) could be analyzed (Table 2).

TABLE 2
Individual Blood Biochemistry Data at the final time point

| Mouse | | | TBIL | ALB | AST | BUN | CK | Hemolysis | Lipemia |
|---|---|---|---|---|---|---|---|---|---|
| ID | Treatment | Group | (mg/dL) | (g/dL) | (U/L) | (mg/dL) | (U/L) | Index | Index |
| WT8.2 | Untreated | 8-week | 0.2 | 3 | 94 | 19 | 298 | Normal | Normal |
| | | interim | | | | | | | |
| WT8.4 | Untreated | 8-week | 0.1 | 2.9 | 60 | 20 | 230 | Normal | Normal |
| | | interim | | | | | | | |
| WT8.5 | Untreated | 8-week | 0.2 | 2.7 | 127 | 16 | 514 | Normal | Normal |
| | | interim | | | | | | | |
| WT15.1 | Untreated | 8-week | 0.2 | 2.7 | 135 | 14 | 449 | Normal | Normal |
| | | interim | | | | | | | |
| WT8.1 | AAV9/hSLC13A5 | 8-week | 0.2 | 2.8 | 75 | 23 | 249 | Normal | Normal |
| | | interim | | | | | | | |
| WT8.3 | AAV9/hSLC13A5 | 8-week | 0.1 | 2.5 | 97 | 16 | 300 | Normal | Normal |
| | | interim | | | | | | | |
| WT15.2 | AAV9/hSLC13A5 | 8-week | 0.2 | 2.9 | 79 | 16 | 284 | Normal | + |
| | | interim | | | | | | | |
| WT9.6 | Untreated | 15 month | 0.1 | 2.8 | 39 | 34 | 101 | Normal | Normal |
| | | end-point | | | | | | | |

There were no treatment related gross tissue abnormalities noted during necropsy. None of the microscopic findings identified in study animals are thought to be treatment-related or otherwise suggestive of adverse effects related to vector administrations in these mice. Any adverse microscopic observations in animals, including glomerulopathy in kidney (Hoane, et al. Toxicology pathology 2016, 44 (5), 687-704) and lymphoma (Ward et al., Experimental and Toxicologic Pathology 2006, 57 (5-6), 377-381) are common incidences in mice of this background strain and age. Further, the incidence of these occurrences between AAV9/hSLC13A5 treated mice and their control sex and age matched littermates was similar (Table 3 and Table 4). Necropsy findings are summarized in 5.

TABLE 3
Histopathology Incidences Count for 8-week Interim Group

| | | | Females (n = 2/group) | | Males (n = 2/group) | |
|---|---|---|---|---|---|---|
| | | | | AAV9/ | | AAV9/ |
| Tissue | Finding | Degree | Untreated | SLC13A5 | Untreated | SLC13A5 |
| Kidneys | Simple dilation of tubules of the renal | Mild | 0 | 2 | 0 | 0 |
| | medulla, not uncommon in mice | | | | | |
| Liver | Multifocal small islands of | Mild | 1 | 2 | 2 | 0 |
| | extramedullary hematopoiesis, | | | | | |
| | expected finding in you mice | | | | | |
| | Multifocal infiltrates with small | Mild | 0 | 0 | 1 | 0 |
| | numbers of mixed inflammatory cell | | | | | |
| | infiltrates with micro-abscess, can | | | | | |
| | occur spontaneously as mice age | | | | | |

1. Numbers represent the number of animals affected in the particular group

TABLE 4
Histopathology Incidences Count for Study End-point Group

| | | | Females (n = 5/group) | | Males (n = 5/group) | |
|---|---|---|---|---|---|---|
| | | | | AAV9/ | | AAV9/ |
| Tissue | Finding | Degree | Untreated | SLC13A5 | Untreated | SLC13A5 |
| Brain | Small area of mineralization | Mild | 0 | 1 | 0 | 0 |
| | in thalamus neuropil | | | | | |
| Thoracic | Perivascular infiltrate with | Mild | 1 | 0 | 0 | 0 |
| Spinal Cord | lymphocytes of a meningeal | | | | | |
| | vessel | | | | | |
| Lumbar | Perivascular infiltrate with | Mild | 1 | 0 | 0 | 0 |
| Spinal Cord | lymphocytes of a meningeal | | | | | |
| | vessel | | | | | |
| Spleen | Variable hemosiderin within | Mild | 4 | 4 | 2 | 3 |
| | the macrophages of the red | | | | | |
| | pulp | | | | | |
| Kidneys | Dilation of tubules of the | Mild to | 2 | 3 | 0 | 1 |
| | renal medulla | Moderate | | | | |
| | Glomerulonephritis | Mild | 1 | 2 | 0 | 1 |
| | Perivascular infiltrates with | Mild | 3 | 3 | 4 | 4 |
| | lymphocytes and plasma | | | | | |
| | cells | | | | | |
| | Mineralized and dilated | Mid | 0 | 0 | 1 | 1 |
| | tubules | | | | | |
| Ovaries | Lymphosarcoma effacing | Mild | 4 | 2 | 0 | 0 |
| | normal parenchyma | | | | | |
| Testes | Multiple vacuoles in a few | Mild | 0 | 0 | 3 | 2 |
| | seminiferous tubules | | | | | |
| Liver | Mild perivascular infiltrates | Mild | 3 | 4 | 3 | 3 |
| | with lymphocytes and | | | | | |
| | plasma cells | | | | | |
| | Multifocal infiltrates with | Mild | 1 | 3 | 3 | 1 |
| | micro-abscess | | | | | |
| | Multifocal lipidosis | Mild | 1 | 1 | 0 | 1 |
| Lung | Perivascular infiltrates with | Mild to | 3 | 3 | 1 | 2 |
| | lymphocytes and plasma | Moderate | | | | |
| | cells | | | | | |
| | Peribronchiolar infiltrates | Mild | 1 | 2 | 0 | 0 |
| | with lymphocytes and | | | | | |
| | plasma cells | | | | | |
| | Mild interstitial pneumonia | Mild | 1 | 1 | 1 | |
| Sciatic | Scattered mast cells | Mild | 3 | 4 | 4 | 1 |
| Nerve | | | | | | |

1. Numbers represent the number of animals affected in each group
2. All abnormalities are considered incidental to age, background strain, metabolic state or environment

TABLE 5
Necropsy Summary for All Study Animals

| | | Age at | | |
|---|---|---|---|---|
| | | Final | | |
| Animal | | procedure | | |
| ID | Sex | (Months) | Treatment | Necropsy Notes |
| WT5.1 | F | 17 | AAV9/SLC13A5 | None |
| WT5.2 | M | 17 | AAV9/SLC13A5 | None |
| WT5.3 | M | 17 | Untreated | None |
| WT5.4 | M | 17 | Untreated | None |
| WT6.1 | F | 17 | Untreated | Heart nicked prior to |
| | | | | perfusion |
| WT6.2 | F | 17 | AAV9/SLC13A5 | None |
| WT6.3 | F | 17 | Untreated | None |
| WT6.4 | F | 17 | Untreated | None |
| WT6.5 | F | 17 | AAV9/SLC13A5 | Liver harvested last |
| WT6.6 | M | 17 | AAV9/SLC13A5 | None |
| WT6.7 | M | 17 | AAV9/SLC13A5 | None |
| WT6.8 | M | 17 | Untreated | None |
| WT8.1 | M | 4 | AAV9/SLC13A5 | None |
| WT8.2 | M | 4 | Untreated | None |
| WT8.3 | M | 4 | AAV9/SLC13A5 | None |
| WT8.4 | M | 4 | Untreated | None |
| WT8.5 | F | 4 | Untreated | None |
| WT8.6 | F | 4 | AAV9/SLC13A5 | None |
| WT9.2 | M | 4 | Untreated | Poor perfusion; spleen |
| | | | | reduced in size |
| WT9.3 | M | 17 | AAV9/SLC13A5 | Poor perfusion |
| WT9.4 | M | 17 | AAV9/SLC13A5 | None |
| WT9.5 | M | 17 | Untreated | None |
| WT15.1 | F | 4 | Untreated | None |
| WT15.2 | F | 4 | AAV9/SLC13A5 | None |
| WT15.3 | F | 17 | AAV9/SLC13A5 | Heart stopped 2 minutes |
| | | | | before blood collection |
| WT16.1 | F | 17 | Untreated | None |
| WT16.2 | F | 17 | Untreated | None |
| WT16.3 | F | 17 | AAV9/SLC13A5 | None |

IV administration of 1×10¹⁴ vg/kg of AAV9/SLC13A5 is safe and well-tolerated in Juvenile WT mice. There were no treatment-related effects observed in either the in-life portion of the study or after microscopic examination of major tissues.

### Example 2: Study to Evaluate the Safety of Intrathecal Dosing of AAV9/hSLC13A5 in 8-Week Old Wild Type C57BL/6 Mice

This study was designed to characterize the toxicity of AAV9/hSLC13A5 in wild type C57BL/6J mice. In an earlier study, an IV dose of 1×10¹⁴ vg/kg was found to be safe for juvenile WT C57BL/6J mice for up to the 15 month time point post vector dosing. This dose of AAV9/hSLC13A5 was well tolerated in spite of transduction of the peripheral tissues post IV dosing with the test article. In this study, a dose of 8×10¹¹ vg/mouse of AAV9/hSLC13A5 was delivered by intrathecal lumbar puncture (IT) in juvenile WT C57BL/6J mice. Animals were then followed up to 15 months post-injection and assessed for toxicity and biodistribution.

### Methods

Wildtype Male and Female C57BL/6J littermates received an IT injection of 8×10¹¹ vg of AAV9/hSLC13A5 (n=9, 5 males, 4 females) or vehicle (n=8, 4 males, 5 females; 350 mM phosphate-buffered saline, 5% sorbitol) at 8 weeks of age. Animals were observed weekly and weighed 3 times per week for 8 weeks post-dosing, then once per week until 6 months post-dosing and then once per month until study endpoint. Animals were aged until 15 months post-dosing and then necropsied for histological and biodistribution analysis. Animals were dosed as juveniles at ˜8 weeks of age and necropsied at 15 months post-dosing or ˜17 months of age.

On the day of injection, mice were randomly selected from each cage without prior knowledge of weight and received an IT injection of vehicle or 8×10¹¹ vg AAV9/hSLC13A5 (Table 6). The volume delivered for each mouse was 5 μL.

TABLE 6
Experimental Design

| | | | Body Weight at | | | Assessments at |
|---|---|---|---|---|---|---|
| | | | Injection (g)^{b} | | Age at | Terminal Endpoint |
| | | Dose | Male | Females | Dosing | (15 Months Post- |
| Group | Route^{a} | (vg/mouse) | (n = 4-5) | (n = 4) | (weeks) | Injection)^{c} |
| 1 | IT | Vehicle | 25.5 ± | 18 ± | 8 | Body weights, clinical |
| | | | 0.9 | 0.5 | | signs, adverse events, |
| | | | | | | mortality, |
| 2 | IT | 8 × 10¹¹ | 23.5 ± | 17.1 ± | 8 | histopathology and |
| | | | 1.5 | 0.3 | | biodistribution |

^{a}IT: intrathecal lumbar puncture injection, a 5 μL dose in vehicle (350 mM phosphate-buggered saline, 5% sorbitol).
^{b}Mean ± SEM.
^{c}N = 4-5 per group assessed for histopathology and n = 3 per group assessed for biodistribution.

Mice were monitored for clinical signs, adverse events, and mortality following the treatment every week.

Mice were weighed 3 times per week for the first 8 weeks, then one time per week until 6 months post-injection and then monthly thereafter. Animals that had lost weight from the previous time point were further observed for motor deficits and malocclusion.

At the study-endpoint (15 months post-dosing), animals were weighed and then deeply anesthetized with an overdose of avertin (0.04 mL/g of a 1.25% solution). Blood was collected as a terminal heart draw. Serum collected at the end of the in-life period was analyzed for blood biochemistry, including total bilirubin (TBIL), albumin (ALB), aspartate aminotransferase (AST), blood urea nitrogen (BUN), and creatine kinase (CK). Levels of these biochemical variables indicate the function of kidney and liver.

Terminal tissue samples were collected for histopathological or biodistribution assessment 15 months following treatment. On the day of necropsy, animals were weighed and then deeply anesthetized as indicated earlier. Blood was collected as a terminal heart draw. Animals intended for histological analysis (n=4 vehicle, n=5 vector) were then perfused with cold PBS containing 1 μg/mL heparin followed by perfusion with 10% NBF. Brain, heart, calf muscle, liver, lung, gonad, spleen, kidney, sciatic nerve, and spine were collected from animals intended for biodistribution analysis (n=3 vehicle, n=3 vector).

Biodistribution was analyzed using quantitative real time PCR (qPCR).

Student's unpaired t-test was used to analyze CBC data. Body weight data were analyzed using Repeat ANOVA, with factors treatment or days-post injection, and followed by with Sidak's multiple comparison test. For all comparisons, statistical significance was set at p<0.05. Data were analyzed and graphed using GraphPad Prism software (v. 9.1.0).

### Results

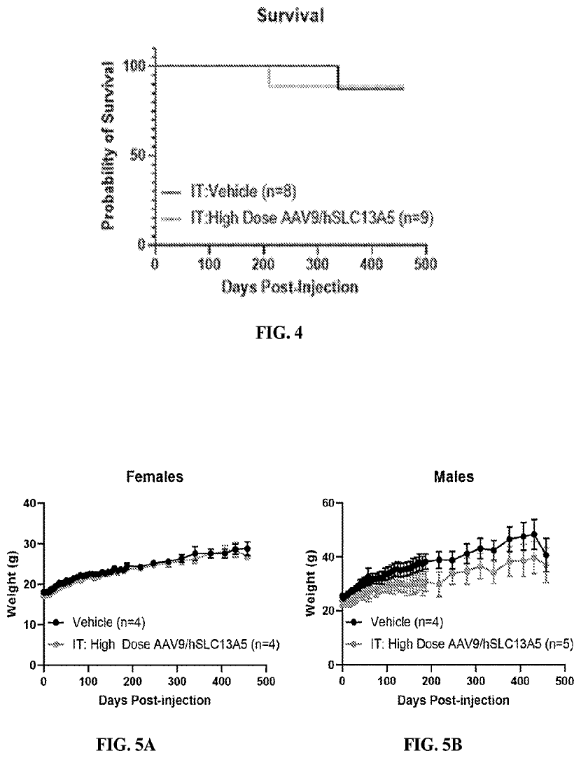

There was one early death in the scAAV9/hSLC13A5 treated group (WT29.4, M) at ˜7 months post-injection and one in the vehicle treated group (WT26.1, F) at ˜11 months post-injection with no significant difference in survival between groups (FIG. 4). Clinical signs of study animals are listed in Table. Malocclusions are not uncommon in the C57BL/6 background strain. For malocclusion mice, their teeth were trimmed weekly.

TABLE 7
Clinical Signs for All Study Animals

| | | | Age at | |
|---|---|---|---|---|
| Animal | | | Death | |
| ID | Sex | Group | (Months)* | Clinical Signs |
| WT26.1 | F | Vehicle | 13 | Animal found 1-2 days post-mortem, |
| | | | | cause of death unknown. |
| WT26.2 | F | scAAV9/hSLC13A5 | 16 | None |
| WT26.3 | M | Vehicle | 16 | None |
| WT27.1 | F | Vehicle | 16 | None |
| WT27.2 | F | scAAV9/hSLC13A5 | 16 | None |
| WT27.3 | F | scAAV9/hSLC13A5 | 16 | None |
| WT27.4 | M | scAAV9/hSLC13A5 | 16 | None |
| WT27.6 | M | scAAV9/hSLC13A5 | 16 | None |
| WT27.7 | M | scAAV9/hSLC13A5 | 16 | Posture and gait noted as abnormal |
| | | | | (Oct. 4, 2019) and natural behavior as |
| | | | | abnormal (Jan. 13, 2020) |
| WT28.1 | F | scAAV9/hSLC13A5 | 16 | None |
| WT28.2 | F | scAAV9/hSLC13A5 | 16 | Malocclusion (Nov. 19, 2020) |
| WT28.3 | M | scAAV9/hSLC13A5 | 16 | None |
| WT28.4 | M | scAAV9/hSLC13A5 | 16 | None |
| WT28.5 | M | scAAV9/hSLC13A5 | 16 | None |
| WT29.1 | F | scAAV9/hSLC13A5 | 16 | None |
| WT29.3 | M | scAAV9/hSLC13A5 | 16 | None |
| WT29.4 | M | scAAV9/hSLC13A5 | 8 | Malocclusion (Sep. 25, 2019). Animal |
| | | | | euthanized early due to penile |
| | | | | prolapse, dehydration, hunched back, |
| | | | | rough coat, difficulty ambulating |
| | | | | (Apr. 8, 2020). |

*Animals dosed at 8 weeks of age.

Body weight was monitored to assess the overall health of the animals. There was no significant difference in body weight between treatment groups within male or female mice (FIGS. 5A and 51B), demonstrating that IT delivered doses of 8×10¹¹ vg was well tolerated in the WT C57BL/6J mice up to 15 months following treatment.

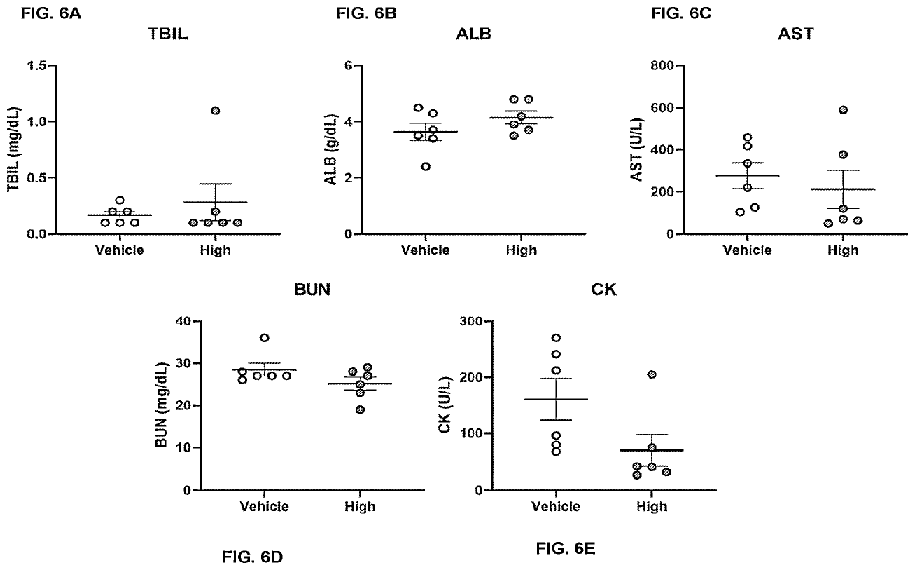

Blood biochemistry analysis from serum did not show significant changes post treatment (FIG. 6A-6E). Individual test results are shown in Table.

TABLE 8
Individual Blood Biochemistry Data

| Mouse | | TBIL | ALB | AST | BUN | CK | Hemolysis | Lipemia |
|---|---|---|---|---|---|---|---|---|
| ID | Treatment | (mg/dL) | (g/dL) | (U/L) | (mg/dL) | (U/L) | Index | Index |
| WT26.03 | Vehicle | 0.1 | 4.3 | 104 | 36 | 80 | Normal | Normal |
| WT27.01 | Vehicle | 0.1 | 3.4 | 126 | 26 | 241 | Normal | Normal |
| WT28.01 | Vehicle | 0.2 | 3.5 | 335 | 27 | 212 | Normal | Normal |
| WT28.03 | Vehicle | 0.1 | 4.5 | 221 | 27 | 96 | Normal | Normal |
| WT29.01 | Vehicle | 0.2 | 2.4 | 459 | 28 | 270 | + | Normal |
| WT29.03 | Vehicle | 0.3 | 3.7 | 417 | 27 | 68 | Normal | Normal |
| WT26.02 | scAAV9/hSLC13A5 | 0.1 | 4.2 | 64 | 23 | 27 | Normal | Normal |
| WT27.02 | scAAV9/hSLC13A5 | 0.1 | 3.5 | 50 | 19 | 32 | Normal | Normal |
| WT27.03 | scAAV9/hSLC13A5 | 0.2 | 3.9 | 71 | 28 | 42 | Normal | Normal |
| WT28.02 | scAAV9/hSLC13A5 | 0.1 | 4.8 | 590 | 25 | 205 | Normal | Normal |
| WT28.04 | scAAV9/hSLC13A5 | 1.1 | 4.8 | 377 | 29 | 75 | + | Normal |
| WT28.05 | scAAV9/hSLC13A5 | 0.1 | 3.7 | 120 | 27 | 41 | Normal | Normal |

Gross tissue abnormalities at necropsy are listed in Table.

TABLE 9
Necropsy Notes for All Study Animals

| | | | Age at | | |
|---|---|---|---|---|---|
| Animal | | | Death | Tissue | |
| ID | Se | Group | (Months) | Analysis | Necropsy Notes |
| WT26.1 | F | Vehicle | 13 | Partial | Animal found 1-2 days |
| | | | | collection | post-mortem and was |
| | | | | | partially cannibalized. |
| | | | | | Brain and spinal cord |
| | | | | | fixed, but not assessed |
| | | | | | due to quality |
| WT26.2 | F | scAAV9/hSLC13A5 | 16 | Histopathology | None |
| WT26.3 | M | Vehicle | 16 | Histopathology | None |
| WT27.1 | F | Vehicle | 16 | Histopathology | None |
| WT27.2 | F | scAAV9/hSLC13A5 | 16 | Histopathology | None |
| WT27.3 | F | scAAV9/hSLC13A5 | 16 | Histopathology | None |
| WT27.4 | M | Vehicle | 16 | Histopathology | None |
| WT27.6 | M | scAAV9/hSLC13A5 | 16 | Histopathology | None |
| WT27.7 | M | scAAV9/hSLC13A5 | 16 | Histopathology | Deformed ribcage, kyphosis, |
| | | | | | small testicles, small |
| | | | | | liver, little body fat |
| WT28.1 | F | Vehicle | 16 | Histopathology | None |
| WT28.2 | F | scAAV9/hSLC13A5 | 16 | Biodistribution | None |
| WT28.3 | M | Vehicle | 16 | Biodistribution | None |
| WT28.4 | M | scAAV9/hSLC13A5 | 16 | Biodistribution | Liver masses, so liver |
| | | | | | piece drop-fixed for |
| | | | | | histopathology |
| WT28.5 | M | scAAV9/hSLC13A5 | 16 | Biodistribution | None |
| WT29.1 | F | Vehicle | 16 | Biodistribution | None |
| WT29.3 | M | Vehicle | 16 | Biodistribution | Liver was small and |
| | | | | | inflamed |
| WT29.4 | M | scAAV9/hSLC13A5 | 8 | Histopathology | Liver was small and |
| | | | | for cause of | yellowish, the spleen |
| | | | | death | small, and kidneys were |
| | | | | | small and jaundice-like |

The microscopic findings identified in study animals do not suggest adverse effects specifically related to vector administrations in these mice. Abnormal histopathology noted in study animals, including hepatocellular carcinoma¹, are common incidences in mice of this background strain and age. Further, the incidence of these occurrences between AAV9/hSLC13A5 treated mice and their control sex and age matched vehicle treated littermates was similar (Table). One exception was the occurrence of dilation of tubules of the renal medulla, which occurred more frequently in AAV9/hSLC13A5 mice. These findings are considered incidental as it is not uncommon in mice and other microscopic abnormalities in the kidneys occurred at an equal frequency between treatment groups.

TABLE 10
Histopathology Incidences Count for Study End-point Animals

| | | | Females | | Males | |
|---|---|---|---|---|---|---|
| | | | | AAV9/ | | AAV9/ |
| | | | Vehicle | SLC13A | Vehicle | SLC13A |
| Tissue | Finding | Degree | (n = 2) | 5 (n = 3) | (n = 2) | 5 (n = 2) |
| Thoracic | Perivascular infiltrate with lymphocytes of a | Mild | | 1 | | |
| Spinal Cord | meningeal vessel, considered incidental | | | | | |
| Lumbar | Perivascular infiltrate with lymphocytes of a | Mild | | 1 | | |
| Spinal Cord | meningeal vessel, considered incidental | | | | | |
| Spleen | Variable hemosiderin within the | Mild | 2 | 2 | 2 | 2 |
| | macrophages of the red pulp, considered | | | | | |
| | normal in adult mice | | | | | |
| Kidneys | Dilation of tubules of the renal medulla, not | Mild to | 1 | 3 | | 1 |
| | uncommon in mice | Moderate | | | | |
| | Perivascular infiltrates with lymphocytes and | Mild | 1 | 2 | 2 | 1 |
| | plasma cells, considered incidental as occurs | | | | | |
| | occasionally in aged mice | | | | | |
| Testes | Multiple vacuoles in a few seminiferous | Mild | | | 1 | |
| | tubules | | | | | |
| | Diffuse tubular degeneration and atrophy | Moderate | | | | 1 |
| | with no evidence of sperm formation | | | | | |
| Liver³ | Mild perivascular infiltrates with | Mild | 2 | 3 | 1 | 1 |
| | lymphocytes and plasma cells, incidental | | | | | |
| | findings as mice age | | | | | |
| | Multifocal infiltrates with micro-abscess, | Mild | 2 | | | 1 |
| | occur spontaneously and more frequently | | | | | |
| | with age | | | | | |
| | Multifocal lipidosis. normal finding | Mild | | 1 | 1 | |
| | depending on metabolic status | | | | | |
| | Extramedullary hematopoiesis, uncommon | Mild | | 1 | 1 | |
| | with aging | | | | | |
| Lung | Perivascular infiltrates with lymphocytes and | Mild to | 2 | 2 | 2 | 1 |
| | plasma cells | Moderate | | | | |
| Sciatic | Scattered mast cells, considered incidental | Mild | 2 | 3 | 2 | 1 |
| Nerve | | | | | | |
| Mass | Hepatocellular carcinoma, occur with age in | Moderate | | | | 1 |
| | this background strain | | | | | |

1. Numbers represent the number of animals affected in each group.
2. “Mild, Mild to moderate” indicate the degree of damage.
³Liver from n = 3 AAV9/SLC13A5 were assessed due to mass for one animal where its other tissues were frozen.

IT administration of 8×10¹¹ vg of AAV9/hSLC13A5 was safe and well-tolerated in juvenile WT mice. There were no treatment-related effects observed in either the in-life portion of the study or after microscopic examination of major tissues.

### Example 3: Study to Evaluate the Safety of AAV9/hSLC13A5 when Administered Intrathecally in Post-Natal Day 10 Wild Type C57BL/6 Mice

This study was designed to characterize the toxicity of AAV9/hSLC13A5 in animals younger than previously tested. In an earlier study (described in Example 2 supra), an IT dose of 8×10¹¹ vg/mouse was found to be safe for juvenile wildtype C57BL/6J mice for up to 12 months post vector dosing. In this study, a low (2×10¹¹ vg/mouse) and high dose (8×10¹¹ vg/mouse) of AAV9/hSLC13A5 was delivered by intrathecal lumbar puncture (IT) on post-natal day 9-10 (P 10) in wildtype C57BL/6J pups.

### Methods

Wildtype male and female C57BL/6J littermates received an IT injection of 2×10¹¹ (n=12, 6 males, 6 females) or 8×10¹¹vg (n=11, 5 males, 6 females) of AAV9/hSLC13A5 or vehicle (n=13, 6 males, 7 females; 350 mM phosphate-buffered saline, 5% sorbitol) at post-natal day 9 or 10 day. Animals were observed weekly and weighed 3 times per week for up to 12 weeks post-dosing, then one time per week. Animals were followed up to 12 months post injection.

### Experimental Design

On the day of injection, mice were randomly selected from each cage without prior knowledge of weight and received an IT injection of vehicle or 2×10¹¹ vg or 8×10¹¹ vg AAV9/hSLC13A5 (Table 4). The volume delivered for each mouse was 5 μL.

TABLE 4
Experimental Design

| | | | Body Weight at | | | |
|---|---|---|---|---|---|---|
| | | | Injection (g)^{b} | | Age at | Terminal Endpoint |
| | | Dose | Male | Female | Dosing (Post- | (12 Months Post- |
| Group | Route^{a} | (vg/mouse) | (n = 6) | (n = 6-7) | natal days) | Injection) |
| 1 | IT | Vehicle | 5.2 ± 0.4 | 4.9 ± 0.1 | 9-10 | Body weights, clinical |
| | | | | | | signs, adverse events |
| 2 | IT | 2 × 10¹¹ | 5.1 ± 0.3 | 5.2 ± 0.3 | 9-10 | mortality, |
| | | | | | | histopathology and |
| 3 | IT | 8 × 10¹¹ | 5.8 ± 0.3 | 5.5 ± 0.3 | 9-10 | biodistribution |

^{a}IT: intrathecal lumbar puncture injection, a 5 μL dose in vehicle (350 mM phosphate-buggered saline, 5% sorbitol).
^{b}Mean ± SEM.

Mice were monitored every week for clinical signs, adverse events, and mortality following treatment. Mice were weighed 3 times per week for up to 12 weeks post-dosing, then one time per week until 10 months post-dosing and then monthly until 12 months post-dosing. Animals that lost weight from the previous time point were further observed for motor deficits and malocclusion.

An 8-week post-dosing interim point blood was collected from the facial vein of all mice on study. Blood was collected at study endpoint as a terminal heart draw. Serum collected at 8 weeks post-dosing and study endpoint were analyzed for blood biochemistry, including total bilirubin (TBIL), albumin (ALB), aspartate aminotransferase (AST), blood urea nitrogen (BUN), and creatine kinase (CK). Levels of these biochemicals indicate the function of kidney and liver.

One-way ANOVA with Tukey's post-hoc analysis was used to analyze clinical blood chemistry data. Body weight data were analyzed using Repeat ANOVA, with factors treatment or days-post injection, and followed by with Tukey's multiple comparison test. For all comparisons, statistical significance was set at p<0.05. Data were analyzed and graphed using GraphPad Prism software (v. 9.1.0).

### Results

There were no early deaths of animals on study (FIG. 7). Clinical signs of study animals have been limited to one vehicle treated animal having a cataract in the remaining eye and the suspected cases of malocclusions. Upon veterinary consultation, all mice were confirmed normal and did not have malocclusion. To date, there have been no signs of toxicity in either vehicle or AAV9/SLC13A5 treated animals.

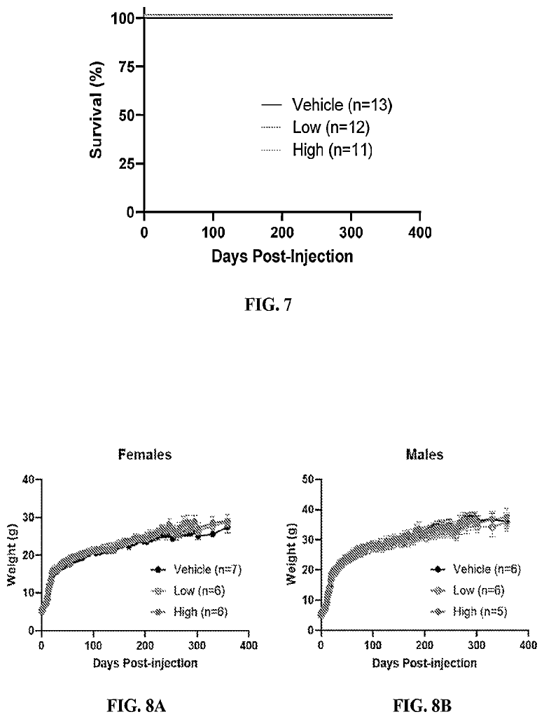

Body weight was monitored to assess the overall health of the animals. Analysis showed no significant difference in body weight between treatment groups within male or female mice (FIGS. 8A and 8B), demonstrating that IT delivered doses up to 8×10¹¹ vg are well tolerated when delivered in wildtype C57BL/6J pups.

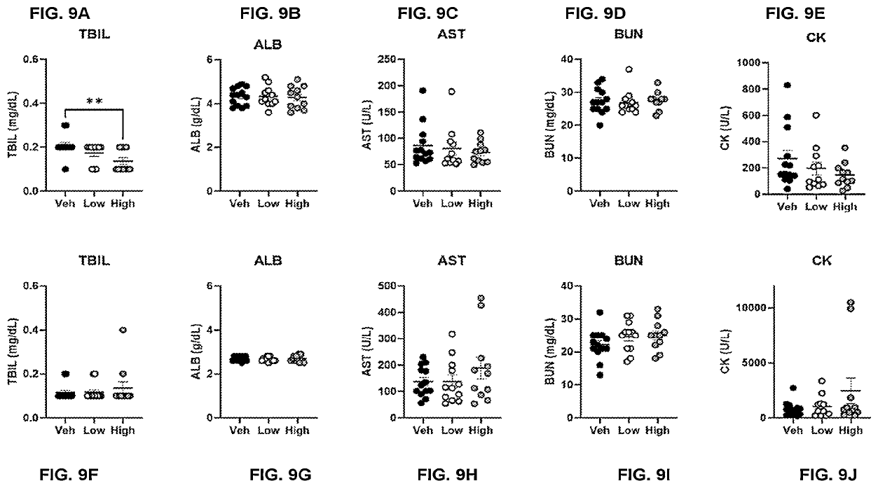

Analysis of blood biochemistry at 8 weeks post vector dosing showed TBIL levels in the high dose treated group were significantly lower than the vehicle treated group (FIG. 9A). While high TBIL levels indicate liver damage, low TBIL levels are not thought to have a negative clinical impact. Blood biochemistry analysis from serum did not show significant changes induced by the treatment in ALB, AST, BUN or CK levels (FIG. 9B-9E). Analysis of blood chemistry at study endpoint showed that TBIL levels normalized in high dose treated animals (FIG. 9F). No significant changes were found 12 months post-dosing in ALB, AST, BUN or CK levels (FIG. 9G-9J).

IT administration of up to 8×10¹¹ vg AAV9/SLC13A5 is safe and well-tolerated in WT pups up to 12 months post-dosing. There were no treatment-related negative effects found during the in-life portion of the study.

### Example 4: Administration of AAV9/hSLC13A5 Intra-Cisterna Magna or Intrathecal to Wild Type and Slc13a5Knockout (KO) Mice

In this non-limiting example, AAV9/hSLC13A5 was delivered intrathecally (IT) or intra-cisterna magna (ICM) at a dose of 2×10¹¹ vg (low dose (LD)) or 8×10¹¹ vg (high dose (HD)) to wild-type and Slc13a5 knockout (KO) mice at about 3 months of age or at P10. Similar to patients, Slc13a5 KO mice have increased plasma citrate levels, EEG abnormalities and an increased susceptibility to seizure induction. Mice were monitored for weight and survival. Blood was collected at baseline and then monthly after treatment and mice received telemetry implants to record baseline EEG and EMG activity. Mice were then tested for susceptibility to seizure induction by pentylenetetrazol (PTZ) and tissues were collected at the study endpoint.

### Results

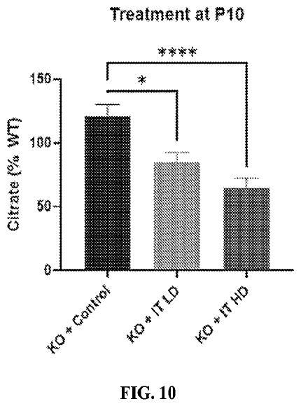

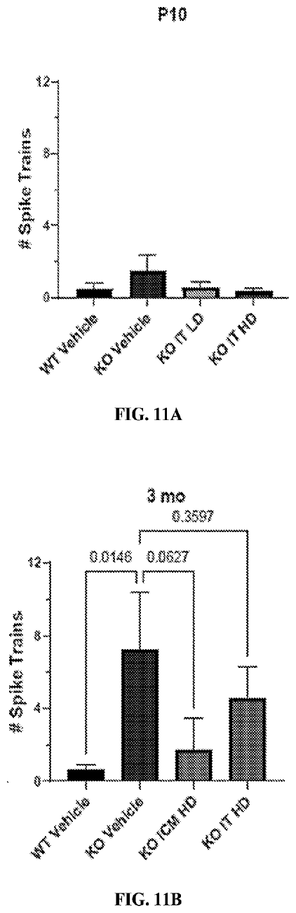

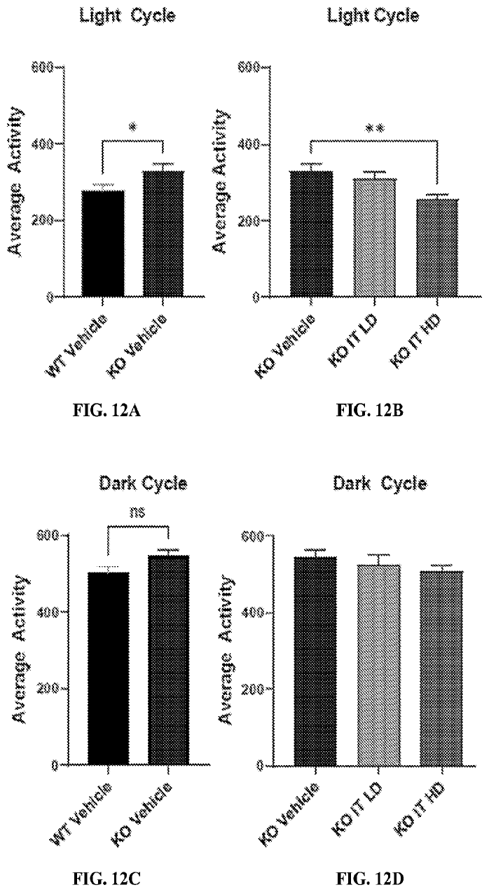

Slc13a5 KO mice treated with scAAV9/SLC13A5 had significantly decreased plasma citrate levels in a dose-dependent manner while Slc13a5 KO mice treated with vehicle had sustained, high citrate levels (FIG. 10). EEG activity was measured using wireless telemetry devices 3 months of age in the P10 treated group and at 8 months of age in the 3 mo treated group. At 3 months of age, epileptic activity was mildly elevated in vehicle treated KO mice and was at WT levels in P10 treated KO mice (FIG. 11A). At 8 months of age, KO mice had significantly higher epileptic activity compared to WT mice, which was normalized with ICM delivery and to a lesser extent with IT delivery when given at 3 months of age (FIG. 11B). General homecage activity was measured in P10 treated mice using wireless telemetry devices over 60 hours. During light cycle/sleep periods KO mice were more active than WT mice, which was normalized with treatment in a dose-dependent manner (FIG. 12A and FIG. 12B). KO mouse dark/awake cycle activity of KO mice trended higher than WT mice and was decreased in a dose-dependent manner with treatment (FIG. 12C and FIG. 12D).

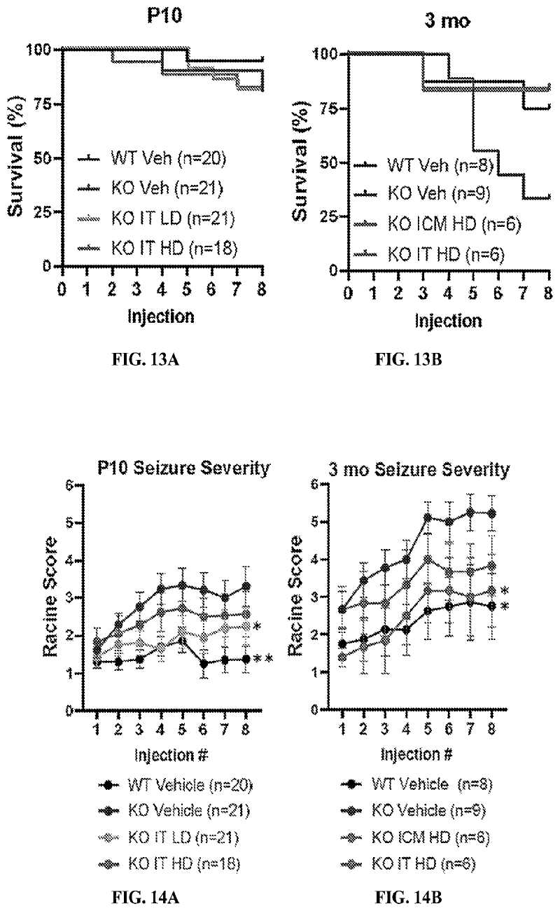

WT and KO mice were injected every other day with 30 mg/kg pentylenetetrazol (PTZ) for up to 8 injections. Mice were observed for 30 minutes post-PTZ injection and assigned a seizure severity score using the modified Racine scale. Latency from time of injection to seizure was also measured. Treatment with AAV9/hSLC13A5 protected against severity-induce death in KO mice. KO mice tested at ˜4 months of age had the same percentage of PTZ induced death as WT mice, which was not affected by treatment at P10 (FIG. 13A). KO mice tested at ˜9 months of age had increased death from seizures as compared to WT, which was rescued with treatment (FIG. 13B).

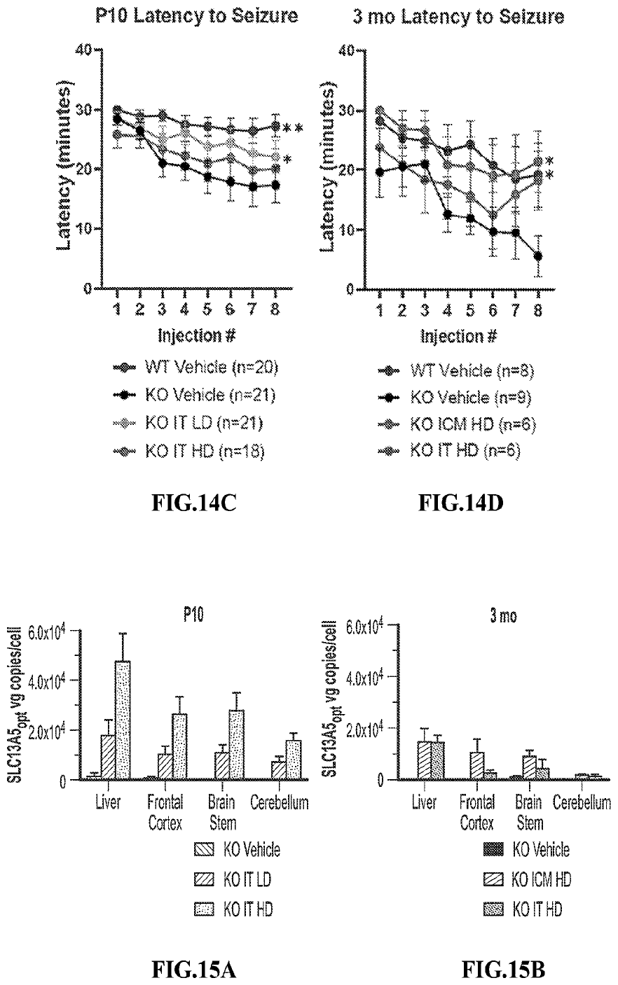

KO controls in both P10 and 3 month old age groups had increased Racine scores compared to WT mice indicating more severe seizures, which was rescued with AAV9/SLC13A5 treatment (FIG. 14A and FIG. 14B). Latency to seizures was significantly reduced in KO vehicle mice compared to WT mice, which was improved with treatment in mice treated at P10 and at 3 months (FIG. 14C and FIG. 14D) Greater benefit was achieved with the LD in the P10 cohort and following ICM delivery in the 3 mo cohort.

Vector biodistribution in treated knockout mice was evaluated. Vector biodistribution in the brain is dose, route and age-dependent. qPCR analysis of DNA from liver and brain regions of treated KO mice showed IT delivery of AAV9/SLC13A5 at P10 resulted in high, wide-spread vector distribution in the brain and liver that was dose-dependent (FIG. 15A). ICM Injection at 3 months resulted in higher and more widespread brain transduction than IT delivery and vector distribution by either route was lower compared to the same dose delivered at P10 (FIG. 15B).

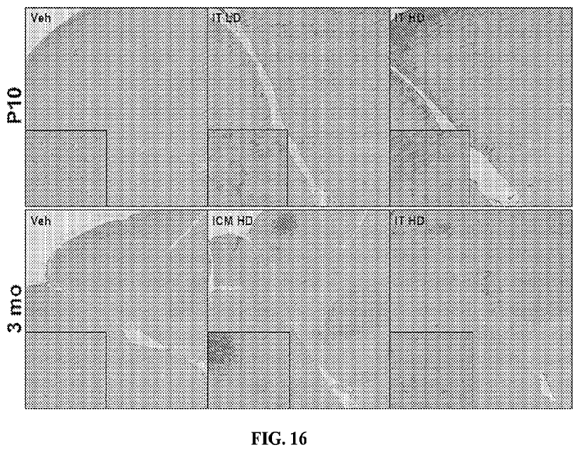

SLC13A5 expression in the brain is dose and route dependent. SLC13A5 immunohistochemical (IHC) staining showed dose-dependent SLC13A5 expression in the brains of mice injected at P10 (FIG. 16 top). In the 3-month study, ICM delivery resulted in greater and more widespread vector expression as compared to IT injected animals (FIG. 16 bottom). Insets show staining consistent with a plasma membrane protein.

Non-GLP toxicology study showed treatment at P10 was well-tolerated up to one year post-injection. KO mice assessed at 3-4 months of age had more normal brain activity and were less susceptible to seizures as compared to older KO mice. Treatment at P10 normalized activity during the sleep cycle and protected KO mice against seizure onset and severity. qPCR analysis showed that vector distribution was dose, route and age-dependent, with IT HD P10 delivery achieving the highest distribution in brain and liver. Brain IHC analysis showed SLC13A5 expression that was dose-dependent in P10 injected mice and route-dependent in 3 mo injected mice. Results support potential safety and benefit of treating with SLC13A5 vector at a younger age and with a lower vector dose than adult mice.

The results summarized in this example demonstrate that the SLC13A5 containing rAAV vectors of the present disclosure can be administered ICM to treat diseases and genetic disorders linked to SLC13A5 loss, misfunction and/or deficiency.

What is claimed is:

1. A recombinant adeno-associated virus (rAAV) vector comprising in 5′ to 3′ direction:
a) a first AAV ITR sequence comprising the sequence of SEQ ID NO: 7;
b) a promoter sequence;
c) a nucleic acid sequence encoding an SLC13A5 polypeptide, wherein the SLC13A5 polypeptide comprises the amino acid sequence set forth in SEQ ID NO: 1;
d) a polyA sequence; and
e) a second AAV ITR sequence comprising the sequence of SEQ ID NO: 8.

2. The rAAV vector of claim 1, wherein the nucleic acid sequence encoding the SLC13A5 polypeptide is a codon optimized nucleic acid sequence.

3. The rAAV vector of claim 1 or 2, wherein the codon optimized nucleic acid sequence encoding a SLC13A5 polypeptide comprises the nucleic acid sequence set forth in SEQ ID NO: 3.

4. The rAAV vector of any one of claims 1-3, wherein the promoter sequence comprises the nucleic acid sequence set forth in SEQ ID NO: 21.

5. The rAAV vector of any one of claims 1-4, wherein the polyA sequence comprises the nucleic acid sequence set forth in SEQ ID NO: 36.

6. The rAAV vector of any one of claims 1-5, wherein the rAAV vector comprises the nucleic acid sequence set forth in SEQ ID NO: 38.

7. An rAAV viral vector comprising:
(i) an AAV capsid protein; and
(ii) an rAAV vector of any one of claims 1-6.

8. The rAAV viral vector of claim 7, wherein the AAV capsid protein is an AAV9 capsid protein.

9. A pharmaceutical composition comprising the rAAV viral vector of claim 7 or 8 and at least one pharmaceutically acceptable excipient and/or additive.

10. A method for treating a subject having a disease and/or disorder involving an SLC13A5 gene, the method comprising administering to the subject at least one therapeutically effective amount of the rAAV viral vector of claim 7 or 8 or the pharmaceutical composition of claim 9.

11. The method of claim 10, wherein the disease and/or disorder involving an SLC13A5 gene is neonatal epileptic encephalopathy.

12. The method of claim 10 or 11, wherein the rAAV viral vector or pharmaceutical composition is administered intrathecally.

13. The method of claim 10 or 11, wherein the rAAV viral vector or pharmaceutical composition is administered intracisterna-magna.


SEQUENCE LISTING (42 sequences)

SEQ ID NO: 1 - protein, 568 aa, Homo sapiens [sequence omitted]

SEQ ID NO: 2 - protein, 568 aa, Homo sapiens [sequence omitted]

SEQ ID NO: 3 - DNA, 1704 bp, Homo sapiens
  source 1..1704: mol_type = other DNA
atggcctcggcgttgagctacgtgtccaagttcaagtcctttgtgatcctgtttgtgacccctctcttgctcctgccactcgtgattctgatgccggccaagtttgtgcgctgcgcctacgtgatcatcctgatggcaatctactggtgtaccgaagtcatccccctcgccgtgacttctcttatgccggtgctgctgttcccgctgttccaaatcctcgacagccgccaggtctgcgtgcagtacatgaaggacaccaacatgctgttcctgggcggcctgattgtggccgtggcagtggagcggtggaacctccacaagcgcatcgccctgaggactctgctctgggtcggcgctaagccagcgcggctgatgctggggttcatgggagtgaccgcgctgctctccatgtggatcagcaacaccgccactaccgccatgatggtcccgatcgtggaagccattctgcaacagatggaagctacttccgccgccactgaagcgggactggaactggtggacaagggaaaggccaaggaactgcccggttcacaagtcatcttcgagggaccaactctgggccagcaggaggaccaagagcgcaagcggctgtgcaaggccatgaccttgtgtatctgctacgccgcttcgatcggaggcactgccaccctgaccgggactggacccaacgtcgtgctgctgggacagatgaacgagctgttcccggattccaaggaccttgtgaacttcgcgtcgtggttcgccttcgcattccctaacatgctcgtgatgctgctctttgcatggctgtggctgcagttcgtgtatatgcggtttaacttcaagaagtcctggggatgcggtctggaatccaagaagaacgagaaggccgcgcttaaggtcctccaggaagaataccggaaattggggccgctgagcttcgccgagatcaacgtgctcatctgtttcttcctgctggtcatcctgtggttctcaagagatcctggtttcatgccggggtggctcactgtggcctgggtcgaaggagaaaccaaatacgtgtcggatgcgactgtggcaatctttgtcgccacgctcctgttcatcgtgccttcccaaaagcctaagttcaacttccgctcccaaaccgaagaggagcgcaaaacaccgttttaccctccacctctgttggactggaaagtcacccaggaaaaggtcccatggggcatcgtgctcctgctgggcggaggtttcgccctggctaagggcagcgaagccagcggtctgtccgtgtggatgggaaaacagatggagccgctgcacgctgtgccccctgctgccatcactctcattctgtccctgcttgtcgccgtgtttaccgagtgcacctccaacgtcgctaccactactctgttcttgcccatcttcgcctcgatgtcgagatccatcggcctgaaccccctctacattatgctgccgtgcaccttgtccgcctccttcgcgttcatgctgcctgtggcgactcccccgaatgctattgtgttcacctacggccatctcaaggtcgcagacatggtcaagaccggagtgatcatgaacattattggcgtgttctgcgtgttcttggccgtgaatacctggggtagagcaattttcgaccttgaccacttcccggactgggccaacgtgacccatatcgaaact

SEQ ID NO: 4 - DNA, 3231 bp, Homo sapiens
  source 1..3231: mol_type = other DNA
ctcactcgtctcgcccgccagtctccctcccgcgcgatggcctcggcgctgagctatgtctccaagttcaagtccttcgtgatcttgttcgtcaccccgctcctgctgctgccactcgtcattctgatgcccgccaagtttgtcaggtgtgcctacgtcatcatcctcatggccatttactggtgcacagaagtcatccctctggctgtcacctctctcatgcctgtcttgcttttcccactcttccagattctggactccaggcaggtgtgtgtccagtacatgaaggacaccaacatgctgttcctgggcggcctcatcgtggccgtggctgtggagcgctggaacctgcacaagaggatcgccctgcgcacgctcctctgggtgggggccaagcctgcacggctgatgctgggcttcatgggcgtcacagccctcctgtccatgtggatcagtaacacggcaaccacggccatgatggtgcccatcgtggaggccatattgcagcagatggaagccacaagcgcagccaccgaggccggcctggagctggtggacaagggcaaggccaaggagctgccagggagtcaagtgatttttgaaggccccactctggggcagcaggaagaccaagagcggaagaggttgtgtaaggccatgaccctgtgcatctgctacgcggccagcatcgggggcaccgccaccctgaccgggacgggacccaacgtggtgctcctgggccagatgaacgagttgtttcctgacagcaaggacctcgtgaactttgcttcctggtttgcatttgcctttcccaacatgctggtgatgctgctgttcgcctggctgtggctccagtttgtttacatgagattcaattttaaaaagtcctggggctgcgggctagagagcaagaaaaacgagaaggctgccctcaaggtgctgcaggaggagtaccggaagctggggcccttgtccttcgcggagatcaacgtgctgatctgcttcttcctgctggtcatcctgtggttctcccgagaccccggcttcatgcccggctggctgactgttgcctgggtggagggtgagacaaagtatgtctccgatgccactgtggccatctttgtggccaccctgctattcattgtgccttcacagaagcccaagtttaacttccgcagccagactgaggaagaaaggaaaactccattttatccccctcccctgctggattggaaggtaacccaggagaaagtgccctggggcatcgtgctgctactagggggcggatttgctctggctaaaggatccgaggcctcggggctgtccgtgtggatggggaagcagatggagcccttgcacgcagtgcccccggcagccatcaccttgatcttgtccttgctcgttgccgtgttcactgagtgcacaagcaacgtggccaccaccaccttgttcctgcccatctttgcctccatgtctcgctccatcggcctcaatccgctgtacatcatgctgccctgtaccctgagtgcctcctttgccttcatgttgcctgtggccacccctccaaatgccatcgtgttcacctatgggcacctcaaggttgctgacatggtgaaaacaggagtcataatgaacataattggagtcttctgtgtgtttttggctgtcaacacctggggacgggccatatttgacttggatcatttccctgactgggctaatgtgacacatattgagacttaggaagagccacaagaccacacacacagcccttaccctcctcaggactaccgaaccttctggcacaccttgtacagagttttggggttcacaccccaaaatgacccaacgatgtccacacaccaccaaaacccagccaatgggccacctcttcctccaagcccagatgcagagatggtcatgggcagctggagggtaggctcagaaatgaagggaacccctcagtgggctgctggacccatctttcccaagccttgccattatctctgtgagggaggccaggtagccgagggatcaggatgcaggctgctgtacccgctctgcctcaagcatcccccacacagggctctggttttcactcgcttcgtcctagatagtttaaatgggaatcggatcccctggttgagagctaagacaaccacctaccagtgcccatgtcccttccagctcaccttgagcagcctcagatcatctctgtcactctggaagggacaccccagccagggacggaatgcctggtcttgagcaacctcccactgctggagtgcgagtgggaatcagagcctcctgaagcctctgggaactcctcctgtggccaccaccaaaggatgaggaatctgagttgccaacttcaggacgacacctggcttgccacccacagtgcaccacaggccaacctacgcccttcatcacttggttctgttttaatcgactggccccctgtcccacctctccagtgagcctccttcaactccttggtcccctgttgtctgggtcaacatttgccgagacgccttggctggcaccctctggggtcccccttttctcccaggcaggtcatcttttctgggagatgcttcccctgccatccccaaatagctaggatcacactccaagtatgggcagtgatggcgctctgggggccacagtgggctatctaggccctccctcacctgaggcccagagtggacacagctgttaatttccactggctatgccacttcagagtctttcatgccagcgtttgagctcctctgggtaaaatcttccctttgttgactggccttcacagccatggctggtgacaacagaggatcgttgagattgagcagcgcttggtgatctctcagcaaacaacccctgcccgtgggccaatctacttgaagttactcggacaaagaccccaaagtggggcaacaactccagagaggctgtgggaatcttcagaacccccctgtaagagacagacatgagagacaagcatcttctttcccccgcaagtccattttatttccttcttgtgctgctctggaagagaggcagtagcaaagagatgagctcctggatggcattttccagggcaggagaaagtatgagagcctcaggaaaccccatcaaggaccgagtatgtgtctggttccttgggtgggacgattcctgaccacactgtccagctcttgctctcattaaatgctctgtctcccgcggaaa

SEQ ID NO: 5 - DNA, 3093 bp, Homo sapiens
  source 1..3093: mol_type = other DNA
ctcactcgtctcgcccgccagtctccctcccgcgcgatggcctcggcgctgagctatgtctccaagttcaagtccttcgtgatcttgttcgtcaccccgctcctgctgctgccactcgtcattctgatgcccgccaagtttgtcaggtgtgcctacgtcatcatcctcatggccatttactggtgcacagaagtcatccctctggctgtcacctctctcatgcctgtcttgcttttcccactcttccagattctggactccaggcaggtgtgtgtccagtacatgaaggacaccaacatgctgttcctgggcggcctcatcgtggccgtggctgtggagcgctggaacctgcacaagaggatcgccctgcgcacgctcctctgggtgggggccaagcctgcacggctgatgctgggcttcatgggcgtcacagccctcctgtccatgtggatcagtaacacggcaaccacggccatgatggtgcccatcgtggaggccatattgcagcagatggaagccacaagcgcagccaccgaggccggcctggagctggtggacaagggcaaggccaaggagctgccagggagtcaagtgatttttgaaggccccactctggggcagcaggaagaccaagagcggaagaggttgtgtaaggccatgaccctgtgcatctgctacgcggccagcatcgggggcaccgccaccctgaccgggacgggacccaacgtggtgctcctgggccagatgaacgagttgtttcctgacagcaaggacctcgtgaactttgcttcctggtttgcatttgcctttcccaacatgctggtgatgctgctgttcgcctggctgtggctccagtttgtttacatgagattcaattttaaaaagtcctggggctgcgggctagagagcaagaaaaacgagaaggctgccctcaaggtgctgcaggaggagtaccggaagctggggcccttgtccttcgcggagatcaacgtgctgatctgcttcttcctgctggtcatcctgtggttctcccgagaccccggcttcatgcccggctggctgactgttgcctgggtggagggtgagacaaagtatgtctccgatgccactgtggccatctttgtggccaccctgctattcattgtgccttcacagaagcccaagtttaacttccgcagccagactgaggaagaaaggaaaactccattttatccccctcccctgctggattggaaggtaacccaggagaaagtgccctggggcatcgtgctgctactagggggcggatttgctctggctaaaggatccgaggcctcggggctgtccgtgtggatggggaagcagatggagcccttgcacgcagtgcccccggcagccatcaccttgatcttgtccttgctcgttgccgtgttcactgagtgcacaagcaacgtggccaccaccaccttgttcctgcccatctttgcctccatggtgaaaacaggagtcataatgaacataattggagtcttctgtgtgtttttggctgtcaacacctggggacgggccatatttgacttggatcatttccctgactgggctaatgtgacacatattgagacttaggaagagccacaagaccacacacacagcccttaccctcctcaggactaccgaaccttctggcacaccttgtacagagttttggggttcacaccccaaaatgacccaacgatgtccacacaccaccaaaacccagccaatgggccacctcttcctccaagcccagatgcagagatggtcatgggcagctggagggtaggctcagaaatgaagggaacccctcagtgggctgctggacccatctttcccaagccttgccattatctctgtgagggaggccaggtagccgagggatcaggatgcaggctgctgtacccgctctgcctcaagcatcccccacacagggctctggttttcactcgcttcgtcctagatagtttaaatgggaatcggatcccctggttgagagctaagacaaccacctaccagtgcccatgtcccttccagctcaccttgagcagcctcagatcatctctgtcactctggaagggacaccccagccagggacggaatgcctggtcttgagcaacctcccactgctggagtgcgagtgggaatcagagcctcctgaagcctctgggaactcctcctgtggccaccaccaaaggatgaggaatctgagttgccaacttcaggacgacacctggcttgccacccacagtgcaccacaggccaacctacgcccttcatcacttggttctgttttaatcgactggccccctgtcccacctctccagtgagcctccttcaactccttggtcccctgttgtctgggtcaacatttgccgagacgccttggctggcaccctctggggtcccccttttctcccaggcaggtcatcttttctgggagatgcttcccctgccatccccaaatagctaggatcacactccaagtatgggcagtgatggcgctctgggggccacagtgggctatctaggccctccctcacctgaggcccagagtggacacagctgttaatttccactggctatgccacttcagagtctttcatgccagcgtttgagctcctctgggtaaaatcttccctttgttgactggccttcacagccatggctggtgacaacagaggatcgttgagattgagcagcgcttggtgatctctcagcaaacaacccctgcccgtgggccaatctacttgaagttactcggacaaagaccccaaagtggggcaacaactccagagaggctgtgggaatcttcagaacccccctgtaagagacagacatgagagacaagcatcttctttcccccgcaagtccattttatttccttcttgtgctgctctggaagagaggcagtagcaaagagatgagctcctggatggcattttccagggcaggagaaagtatgagagcctcaggaaaccccatcaaggaccgagtatgtgtctggttccttgggtgggacgattcctgaccacactgtccagctcttgctctcattaaatgctctgtctcccgcggaaa

SEQ ID NO: 6 - DNA, 3180 bp, Homo sapiens
  source 1..3180: mol_type = other DNA
ctcactcgtctcgcccgccagtctccctcccgcgcgatggcctcggcgctgagctatgtctccaagttcaagtccttcgtgatcttgttcgtcaccccgctcctgctgctgccactcgtcattctgatgcccgccaagtttgtcaggtgtgcctacgtcatcatcctcatggccatttactggtgcacagaagtcatccctctggctgtcacctctctcatgcctgtcttgcttttcccactcttccagattctggactccaggcaggtgtgtgtccagtacatgaaggacaccaacatgctgttcctgggcggcctcatcgtggccgtggctgtggagcgctggaacctgcacaagaggatcgccctgcgcacgctcctctgggtgggggccaagcctgcacgtaacacggcaaccacggccatgatggtgcccatcgtggaggccatattgcagcagatggaagccacaagcgcagccaccgaggccggcctggagctggtggacaagggcaaggccaaggagctgccagggagtcaagtgatttttgaaggccccactctggggcagcaggaagaccaagagcggaagaggttgtgtaaggccatgaccctgtgcatctgctacgcggccagcatcgggggcaccgccaccctgaccgggacgggacccaacgtggtgctcctgggccagatgaacgagttgtttcctgacagcaaggacctcgtgaactttgcttcctggtttgcatttgcctttcccaacatgctggtgatgctgctgttcgcctggctgtggctccagtttgtttacatgagattcaattttaaaaagtcctggggctgcgggctagagagcaagaaaaacgagaaggctgccctcaaggtgctgcaggaggagtaccggaagctggggcccttgtccttcgcggagatcaacgtgctgatctgcttcttcctgctggtcatcctgtggttctcccgagaccccggcttcatgcccggctggctgactgttgcctgggtggagggtgagacaaagtatgtctccgatgccactgtggccatctttgtggccaccctgctattcattgtgccttcacagaagcccaagtttaacttccgcagccagactgaggaagaaaggaaaactccattttatccccctcccctgctggattggaaggtaacccaggagaaagtgccctggggcatcgtgctgctactagggggcggatttgctctggctaaaggatccgaggcctcggggctgtccgtgtggatggggaagcagatggagcccttgcacgcagtgcccccggcagccatcaccttgatcttgtccttgctcgttgccgtgttcactgagtgcacaagcaacgtggccaccaccaccttgttcctgcccatctttgcctccatgtctcgctccatcggcctcaatccgctgtacatcatgctgccctgtaccctgagtgcctcctttgccttcatgttgcctgtggccacccctccaaatgccatcgtgttcacctatgggcacctcaaggttgctgacatggtgaaaacaggagtcataatgaacataattggagtcttctgtgtgtttttggctgtcaacacctggggacgggccatatttgacttggatcatttccctgactgggctaatgtgacacatattgagacttaggaagagccacaagaccacacacacagcccttaccctcctcaggactaccgaaccttctggcacaccttgtacagagttttggggttcacaccccaaaatgacccaacgatgtccacacaccaccaaaacccagccaatgggccacctcttcctccaagcccagatgcagagatggtcatgggcagctggagggtaggctcagaaatgaagggaacccctcagtgggctgctggacccatctttcccaagccttgccattatctctgtgagggaggccaggtagccgagggatcaggatgcaggctgctgtacccgctctgcctcaagcatcccccacacagggctctggttttcactcgcttcgtcctagatagtttaaatgggaatcggatcccctggttgagagctaagacaaccacctaccagtgcccatgtcccttccagctcaccttgagcagcctcagatcatctctgtcactctggaagggacaccccagccagggacggaatgcctggtcttgagcaacctcccactgctggagtgcgagtgggaatcagagcctcctgaagcctctgggaactcctcctgtggccaccaccaaaggatgaggaatctgagttgccaacttcaggacgacacctggcttgccacccacagtgcaccacaggccaacctacgcccttcatcacttggttctgttttaatcgactggccccctgtcccacctctccagtgagcctccttcaactccttggtcccctgttgtctgggtcaacatttgccgagacgccttggctggcaccctctggggtcccccttttctcccaggcaggtcatcttttctgggagatgcttcccctgccatccccaaatagctaggatcacactccaagtatgggcagtgatggcgctctgggggccacagtgggctatctaggccctccctcacctgaggcccagagtggacacagctgttaatttccactggctatgccacttcagagtctttcatgccagcgtttgagctcctctgggtaaaatcttccctttgttgactggccttcacagccatggctggtgacaacagaggatcgttgagattgagcagcgcttggtgatctctcagcaaacaacccctgcccgtgggccaatctacttgaagttactcggacaaagaccccaaagtggggcaacaactccagagaggctgtgggaatcttcagaacccccctgtaagagacagacatgagagacaagcatcttctttcccccgcaagtccattttatttccttcttgtgctgctctggaagagaggcagtagcaaagagatgagctcctggatggcattttccagggcaggagaaagtatgagagcctcaggaaaccccatcaaggaccgagtatgtgtctggttccttgggtgggacgattcctgaccacactgtccagctcttgctctcattaaatgctctgtctcccgcggaaa

SEQ ID NO: 7 - DNA, 106 bp, synthetic construct
  source 1..106: mol_type = other DNA
ctgcgcgctcgctcgctcactgaggccgcccgggcaaagcccgggcgtcgggcgacctttggtcgcccggcctcagtgagcgagcgagcgcgcagagagggagtgg

SEQ ID NO: 8 - DNA, 169 bp, synthetic construct
  source 1..169: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttc

SEQ ID NO: 9 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 10 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 11 - DNA, 168 bp, synthetic construct
  source 1..168: mol_type = other DNA
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatct

SEQ ID NO: 12 - DNA, 168 bp, synthetic construct
  source 1..168: mol_type = other DNA
agatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaccccccccccccccccc

SEQ ID NO: 13 - DNA, 170 bp, synthetic construct
  source 1..170: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttc

SEQ ID NO: 14 - DNA, 104 bp, synthetic construct
  source 1..104: mol_type = other DNA
tgcgcgctcgctcgctcactgaggccgcccgggcaaagcccgggcgtcgggcgacctttggtcgcccggcctcagtgagcgagcgagcgcgcagagagggagtg

SEQ ID NO: 15 - DNA, 106 bp, synthetic construct
  source 1..106: mol_type = other DNA
ctgcgcgctcgctcgctcactgaggccgccggcccaaagggccggcgtcgggcgacctttggtcgcccggcctcagtgagcgagcgagcgcgcagagagggagtgg

SEQ ID NO: 16 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgccggccctttgggccggcggcctcagtgagcgagcgagcgcgcagagagggagtggccaa

SEQ ID NO: 17 - DNA, 145 bp, synthetic construct
  source 1..145: mol_type = other DNA
aggaacccctagtgatggagttggccactccctctctgcgcgctcgctcgctcactgaggccgccggcccaaagggccggcgtcgggcgacctttggtcgcccggcctcagtgagcgagcgagcgcgcagagagggagtggccaa

SEQ ID NO: 18 - DNA, 106 bp, synthetic construct
  source 1..106: mol_type = other DNA
ccactccctctctgcgcgctcgctcgctcactgaggccgggcgaccaaaggtcgcccgacgcccgggctttgcccgggcggcctcagtgagcgagcgagcgcgcca

SEQ ID NO: 19 - DNA, 426 bp, synthetic construct
  source 1..426: mol_type = other DNA
ataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaa

SEQ ID NO: 20 - DNA, 164 bp, synthetic construct
  source 1..164: mol_type = other DNA
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgt

SEQ ID NO: 21 - DNA, 332 bp, synthetic construct
  source 1..332: mol_type = other DNA
ggtaccgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacag

SEQ ID NO: 22 - DNA, 225 bp, synthetic construct
  source 1..225: mol_type = other DNA
caattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaa

SEQ ID NO: 23 - DNA, 796 bp, synthetic construct
  source 1..796: mol_type = other DNA
tacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgga

SEQ ID NO: 24 - DNA, 241 bp, synthetic construct
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 25 - DNA, 448 bp, synthetic construct
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 26 - DNA, 469 bp, synthetic construct
  source 1..469: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 27 - DNA, 302 bp, synthetic construct
  source 1..302: mol_type = other DNA
acgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgg

SEQ ID NO: 28 - DNA, 257 bp, synthetic construct
  source 1..257: mol_type = other DNA
tacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatct

SEQ ID NO: 29 - DNA, 93 bp, synthetic construct
  source 1..93: mol_type = other DNA
ggagtcgctgcgcgctgccttcgccccgtgccccgctccgccgccgcctcgcgccgcccgccccggctctgactgaccgcgttactcccacag

SEQ ID NO: 30 - DNA, 43 bp, synthetic construct
  source 1..43: mol_type = other DNA
gtgagcgggcgggacggcccttctcctccgggctgtaattagc

SEQ ID NO: 31 - DNA, 92 bp, synthetic construct
  source 1..92: mol_type = other DNA
aagaggtaagggtttaagggatggttggttggtggggtattaatgtttaattacctggagcacctgcctgaaatcactttttttcaggttgg

SEQ ID NO: 32 - DNA, 10 bp, synthetic construct
  source 1..10: mol_type = other DNA
gccaccatgg

SEQ ID NO: 33 - DNA, 123 bp, synthetic construct
  source 1..123: mol_type = other DNA
tgtttattgcagcttataatggttacaaataaagcaatagcatcacaaatttcacaaataaagcatttttttcactgcattctagttgtggtttgtccaaactcatcaatgtatcttatcatg

SEQ ID NO: 34 - DNA, 254 bp, synthetic construct
  source 1..254: mol_type = other DNA
ctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatggcttctgaggcggaaagaaccagct

SEQ ID NO: 35 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatgg

SEQ ID NO: 36 - DNA, 59 bp, synthetic construct
  source 1..59: mol_type = other DNA
tcgagaggcctaataaagagctcagatgcatcgatcagagtgtgttggttttttgtgtg

SEQ ID NO: 37 - DNA, 586 bp, synthetic construct
  source 1..586: mol_type = other DNA
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccg

SEQ ID NO: 38 - DNA, 2420 bp, synthetic construct
  source 1..2420: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcggtaccgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagtccggaaccggtgccaccatggcctcggcgttgagctacgtgtccaagttcaagtcctttgtgatcctgtttgtgacccctctcttgctcctgccactcgtgattctgatgccggccaagtttgtgcgctgcgcctacgtgatcatcctgatggcaatctactggtgtaccgaagtcatccccctcgccgtgacttctcttatgccggtgctgctgttcccgctgttccaaatcctcgacagccgccaggtctgcgtgcagtacatgaaggacaccaacatgctgttcctgggcggcctgattgtggccgtggcagtggagcggtggaacctccacaagcgcatcgccctgaggactctgctctgggtcggcgctaagccagcgcggctgatgctggggttcatgggagtgaccgcgctgctctccatgtggatcagcaacaccgccactaccgccatgatggtcccgatcgtggaagccattctgcaacagatggaagctacttccgccgccactgaagcgggactggaactggtggacaagggaaaggccaaggaactgcccggttcacaagtcatcttcgagggaccaactctgggccagcaggaggaccaagagcgcaagcggctgtgcaaggccatgaccttgtgtatctgctacgccgcttcgatcggaggcactgccaccctgaccgggactggacccaacgtcgtgctgctgggacagatgaacgagctgttcccggattccaaggaccttgtgaacttcgcgtcgtggttcgccttcgcattccctaacatgctcgtgatgctgctctttgcatggctgtggctgcagttcgtgtatatgcggtttaacttcaagaagtcctggggatgcggtctggaatccaagaagaacgagaaggccgcgcttaaggtcctccaggaagaataccggaaattggggccgctgagcttcgccgagatcaacgtgctcatctgtttcttcctgctggtcatcctgtggttctcaagagatcctggtttcatgccggggtggctcactgtggcctgggtcgaaggagaaaccaaatacgtgtcggatgcgactgtggcaatctttgtcgccacgctcctgttcatcgtgccttcccaaaagcctaagttcaacttccgctcccaaaccgaagaggagcgcaaaacaccgttttaccctccacctctgttggactggaaagtcacccaggaaaaggtcccatggggcatcgtgctcctgctgggcggaggtttcgccctggctaagggcagcgaagccagcggtctgtccgtgtggatgggaaaacagatggagccgctgcacgctgtgccccctgctgccatcactctcattctgtccctgcttgtcgccgtgtttaccgagtgcacctccaacgtcgctaccactactctgttcttgcccatcttcgcctcgatgtcgagatccatcggcctgaaccccctctacattatgctgccgtgcaccttgtccgcctccttcgcgttcatgctgcctgtggcgactcccccgaatgctattgtgttcacctacggccatctcaaggtcgcagacatggtcaagaccggagtgatcatgaacattattggcgtgttctgcgtgttcttggccgtgaatacctggggtagagcaattttcgaccttgaccacttcccggactgggccaacgtgacccatatcgaaacttgataggcggccgcggagctctcgagaggcctaataaagagctcagatgcatcgatcagagtgtgttggttttttgtgtgacgcgtaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttc

SEQ ID NO: 39 - DNA, 105 bp, synthetic construct
  source 1..105: mol_type = other DNA
cgcggaacccctatttgtttatttttctaaatacattcaaatatgtatccgctcatgagacaataaccctgataaatgcttcaataatattgaaaaaggaagagt

SEQ ID NO: 40 - DNA, 816 bp, synthetic construct
  source 1..816: mol_type = other DNA
atgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 41 - DNA, 29 bp, synthetic construct
  source 1..29: mol_type = other DNA
ttcaaatatgtatccgctcatgagacaat

SEQ ID NO: 42 - DNA, 1602 bp, synthetic construct
  source 1..1602: mol_type = other DNA
actctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggct